SEQUENCE LISTING (79 sequences)

SEQ ID NO: 1 - DNA, 7959 bp, Homo sapiens
atgggcaccaggggagcggtcatgccccctcctatgtgggggctgctgggctgctgttttgtctgtgcctgggctctggggggtccacggcccatccgctctctgccccctctgtcttcccaagtcaagccaggatctgtacccatgcaggtgcccctagagggggccgaggccgccctcgcttatctctactctggagatgcccagcagctatcacaggtgaattgcagtgagcgctatgaagcgcgtggggcaggagccatgccagggctccccccaagcctacagggggcagcgggcacccttgcccaggccgccaattttctcaacatgctgctgcaagccaacgacatccgtgagtccagtgtggaggaggatgtggaatggtaccaggcactggtccgcagcgtggccgagggggacccaagagtgtacagggctttgctgacctttaaccctccaccaggggccagccacctacagctggccctgcaggccacccggactggggaggaaaccatcctgcaggacttgtctgggaactgggtgcaggaggagaaccctcctggggacctggacacccctgccctgaagaagcgagtgttgaccaatgacctagggagcctcggcagccccaagtggccgcaggcagatggatatgtgggggacacgcagcaggtgaggctgtctcctcctttcctggaatgccaggagggacggctccgacctggatggctgatcacactctctgccaccttctatggactcaagccagacctcagcccagaagtcagggggcaggtgcagatggacgtagatctccagagtgtggacatcaatcagtgtgcaagtggcccaggctggtactctaacacacacctgtgtgatctcaacagcacccagtgtgttcccctggagagtcagggctttgttcttggccgctacctctgccgctgccgacctggattctacggggcaagcccctctggggggttagaggagagtgacttccagactaccgggcaattcgggttcccagaaggcagatctgggagactgctgcagtgtctgccatgtcctgagggctgcaccagctgcatggatgccacaccgtgcctggtggaagaggccgcggtgctgcgggccgctgtgctggcctgccaggcctgctgcatgctggccatcttcctgagcatgctggtctcctaccgctgccgccggaacaagaggatctgggcatctggagtggtcctgctggaaactgtcctttttggattcctgctgctttactttcctgtcttcatcctatacttcaagcccagtgtattccgctgcatcgctcttcgctgggtgcggctgctgggttttgccatcgtctacggcaccatcatactcaagctttacagagtgctgcagctgtttctgtctcgaacggcccagcggagtgcccttctgagcagcgggcggctgctgcggcgcctggggctgctcctgctacctgtgctgggcttcctggctgtgtggaccgtgggcgccctggagcgaggcatccagcacgcacctctggtgatccgaggccacactcccagtggccgccatttctacctctgtcaccacgaccgctgggactacatcatggttgtggctgagctgctgctgctgtgctggggcagcttcctctgctacgccacacgggctgtgctctcggccttccatgagccacgctacatgggcatcgccctgcacaatgagctactgctttccgctgccttccacacagccaggtttgtgctggttccctctctgcacccggactggaccctcctcctcttcttcttccacacccacagcacagtcaccaccacgctggctctgatcttcatccctaagttctggaagctgggggctcctccccgggaggagatggtggatgaggtgtgtgaggacgagctggacctgcagcactcaggctcctaccttggcagcagcatcgcctcagcctggagtgagcacagcctggaccctggagacattcgggacgagctgaagaagctctatgcccagctagaggtccacaaaaccaaggaaatggccgcaaacaacccccacctgcccaagaagcgaggcagctcatgccagggactgggccgctccttcatgaggtacctggcggaattccccgaggccctggccaggcagcactcccgggactcaggatccccaggccacggcagcctgcccggctcctcccgccgccggctcctcagctccagcctccaggaacccgaggggacaccagctctgcacaagtcccgcagcacctatgaccagcgcagggagcaggacccgcctcttcttgactcactgctgaggaggaagctggccaagaaggcctctcgaacagagagccgggagtcggtggaggggccccctgccctgggcttcaggtcagccagcgcccacaacctgacggtgggagagaggctacccagagcccggcccgcctctctgcagaagtcgctcagtgtggccagctccagggaaaaggccttgctcatggccagccaggcctacctggaggagacctaccggcaagcaaaggagcgggaggagcggaagaaggccaaggcagccatggccagcctggtgcggaggccatcagccaggaggctggagcggcctcgaggggcccccctgtcagctccaccttcccctgccaagagcagcagcgtggacagctctcacacctctgggaggcttcatgaggaggctaggagaaggctgcctcatccacccatcaggcaccaggtttctacccccatcttggccctgtctgggggcctgggagagccaaggatgctatctcccacctccaccttggctccagctctgctgccagctctagctccaaccccagcccctgccctggcaccagtcccagtatccccacaaagccccaacttactcacctacatctgcccctgggagaacgcagaactgccagccaagcaagaaaatgtgccccaggaaggcccctcagggccagagcgaggccaccactcccctgccccagctcgagccaggctctggagggccctctctgttgcagtagagaaaagcagggctggggagaatgagatggacgcagaggatgcacatcaccagagggaagctaatgatgtggacgaagacaggcccaagatcttccctaaatcccacagcctcaaggcccctgttcagcagggttccatgcgcagcctggggctggcgattaaagctctgacccgttctcggagcacctacagagagaaggagagtgtggaggagagtcccgaggggcagaacagcgggactgcgggagagagtatgggggcaccctcccgatcgcccaggctaggccggcccaaggcggtgagtaagcaggccgctcttatcccctccgatgacaaggagtccctccagaaccaacagaacgctcacaccagcaggatgctccaagtctgtcaacgggagggcagcagggaacaagaagacagaggcaggaggatgacccagggtctaggggaacggaaagctgagagagcaggtaaaacagggcttgccatgctgaggcaagtttccagggacaaaaacatcaagcaatcaaaagaaacccctgtcgggtggcaggaactgcccaaagctggcctccagtccctcggcagcgctgaccacagggtggcagaggtatgcccctgggaggtcactgaatcagaaacgcgtcagccagacagtggcaacaaggccgaaatctgcccctgggagacgagtgaaggagccccagagtcgagggcactaagacaagacccaggtgactcccaaaaaaagagaggggaggcccggggaaaatcagagcccatagatgtggttcccatgatgcggaaaaagccagagaggctggtgagggagcaggaagcagtgtgtccctgggagagtgccgatcgaggaggtctgtcccctgggtcagctcctcaggaccctggcagaatcagagacaaatctgaggcgggggacagtgtggaggccaggaaggtggagaagcctgggtgggaagctgctggcccagaagctcatacccctgacatcaccaaggcagagccgtgtccctgggaggcaagtgaaggaggcgaggatgggaaaccagcccaagaggcagtgaaggatctccctcaggaaaagcagaaaaccaggaaagcaaccttttggaaagaacagaaaccgggaggagacttggagtctctttgtccatgggagagtacagatttccggggcccctcagcagtctcaattcaggccccaggaagctcagagtgttcagggagtttgggcagtggcattgctgaagtgtgtctgtgggaggcaggagatgctcctgctatccagaaagcagagatctgtccctgggagctggatgataacgtgatggggcaggaaatgctgagtctggggacaggtagagaatctcttcaagaaaaggaaaaagcctccagaaaaggaagctttggagagatgggggaacaaactgtgaaagcagtgcagaaattaagtcaacagcaggagtcagtttgtcccagggagagcacggtccctgggcactccagcccatgtctagacaattcctcatccaaagctggtagccaattcctatgcaatggaggaagcagagcaacgcaggtgtgtccacaggaagatctcaggccggaggcacaggaagcaacacctgccaaaacagaaatctgtccctgggaggtaaatgaaagaacaagagaggaatggacatcagcacaggtgccaagaggaggagaatctcaaaaggacaaggagaaaatgcctggaaaatcggaaatcgaagatgtcacagcttgggaaaagcctgaggggcagatccaaaagcaagaagcggtcggcccctgggagagtgtggaccctggcagcttctccccacaaccacgtcctcaagacacagagagaccccaaacccttctccagatgtcaggcagtgtgggaagcaaagctgccgacatttgccctttggatgtggaggaaaacttgactgctgggaaggcagaaatctgtccctgggaggtgggtgctggagcaggggaggaaagggctttgggagctgaggccattaggaaatctccaaatgatacaggcaaggtttctgcagatcttggacccagggagagagctgttactgctccagagaagccacagaagccaaccccagagtgggaggtggcttgtccctgggggagtgtgggtccaggggcctgttctcagcatccaggtactctagatgctgatggaccaaaagctgggttccaggaactggatcatatgggctgcaggccaggtgaagtgtgtccctgggaagcacaggaagctgctaccagtgaaaaagccaagatctgtccctgggaggtaagtgaaggaactactgggaagggattggaccaaaaggcagggagtgaatcagcagagcagagggagaaagctctagaaaaggggagactcacttccctgggagaagacgtatcaaaagggatggcaaaactgtgtcaacaacaggaaactatttgtatttgggagaacaaggacttgagggaatcccctgctcaggcccccaagatctcagacttgcccagcagcatgagtagtgaagtggcagagggacattccttggaagcaacagagaagggggacctgagacaagacccaaagacaggttccttcccagaacacataacccaagaaaaagctccagctgcagacacagaagaattcactactgaagatggggaaaaaacaagccatgagctacaatccgtctgtccatgggagaccactgccccagcagattccgtctctcacctagacagacagcgccctgaccaacctaaagctagctcccagagactggtcagcactgggggcagggccgctgacgtgtgcccatgggatgttcctgatgcaggtgtgtataaatctgacagcagtgccaaggctgagacctgtccctgggaagtgactgaaagaatccctgtcaaaggggtgtcaaggcaggatggaaaaggggactctcaagaagagaaaggcagagccccagaaaaatcagagccaaaaggtgtgccagttcagaaaaagccagagatggcagacttcaggcagcaggaggctgtgtgtccctgggagagtcaagatggcaagggtctgtccccacagccagccccagatgcttctgacagaagcagaggcagttctgaggcagcaggcagtgtggagaccagggtagcggaagtgtgtctgtgggaagtggtagaggctccctctgccaagaaagcagagatctgcccttgggaggcgggtggaggagcagcagaggaaggggaacaggaaagagaatcacaagggcaaggagagatgttccttcagaaggcaggacctggagggacggaagaacacttctcaaaagcagcagcaaagcccagagagcaggaggcagtctgccctggggaaggcacaggctcaggagggctcttgccccagtcaggtgccctggacccagaactcaaagtcagccccaaggaagcaggcagcatgggaagcaggatggcagagctgtgccaatgggaaatcacagatccagaaggaaataaaataaagggtaccatggcagacatctgtcctggggaggaaactggagtcccatctgaggaatctggcctcctggctttaacagcaactcggagagaatttttccccacagctcctgaaaaaccactatgccttttagtccatgggcctctggatcacttctttccagaaagcaaaatcccctgccccaaggtaagcaggccagccagtactttcactctagaaggtgtcagagaactacaaggaccttcagggcttgagccaaggaccagcttagccccagagccaagtctccaggaagctgagtctcagtcttcgtccttaactgaagactcaggccaagtggcttttgaagctcagtatgaagaattcacccctcccactgtctatccttgggattgggagtaacagccttattaggtgaggtcaaacacagagctagctttcaggagccaaggccctttccaagtccaggtgttcccaaagagctgaatcaaagacagcttggccacttcccctccttgagaagaccagaagtcaacccatcccaaagacaaactgcatgaaaagggtacagcttagaccccaatgggaaggcccaccctctctttacttctaacttttcttcctgtttgaggaaacaaagactggacactctacttctaaggacgcctcccatccacctacagtactcacaacacaaaccacccgagtctggacactcacccacaaagccactgttagggatgaaaaaagtcagttgtgcccagcctaccttctctcacagggggacccaattctctgaagtctacgatggagcattttttgagaagaaactaaaccatacaaagtgttggcaacgtagggatggtggaccacagaaggctataaaggccaccatagaaagctgaagagccatttaattgaagcggaagataaattgggggaaaagctaaaatgactcaaaataggggcctgaaaatgctttaaggaccttgtcattttgactttgaatctcctgtttctgctttattgcttctcatacctgccctgcttgactgctgaagcagaaacaatcaggcacgctgacacccttatcagcatctggcatccaaagcaacatcagagagagagaacacagaagaaaagcaattgccccaggtcctccccacagtcaagggagctcttccttccagacatccttgtgtgcctcttgctagttttttagggggtctccccctcttcctgccctagctctcacca

SEQ ID NO: 2 - protein, 2367 aa, Homo sapiens
MGTRGAVMPPPMWGLLGCCFVCAWALGGPRPIRSLPPLSSQVKPGSVPMQVPLEGAEAALAYLYSGDAQQLSQVNCSERYEARGAGAMPGLPPSLQGAAGTLAQAANFLNMLLQANDIRESSVEEDVEWYQALVRSVAEGDPRVYRALLTFNPPPGASHLQLALQATRTGEETILQDLSGNWVQEENPPGDLDTPALKKRVLTNDLGSLGSPKWPQADGYVGDTQQVRLSPPFLECQEGRLRPGWLITLSATFYGLKPDLSPEVRGQVQMDVDLQSVDINQCASGPGWYSNTHLCDLNSTQCVPLESQGFVLGRYLCRCRPGFYGASPSGGLEESDFQTTGQFGFPEGRSGRLLQCLPCPEGCTSCMDATPCLVEEAAVLRAAVLACQACCMLAIFLSMLVSYRCRRNKRIWASGVVLLETVLFGFLLLYFPVFILYFKPSVFRCIALRWVRLLGFAIVYGTIILKLYRVLQLFLSRTAQRSALLSSGRLLRRLGLLLLPVLGFLAVWTVGALERGIQHAPLVIRGHTPSGRHFYLCHHDRWDYIMVVAELLLLCWGSFLCYATRAVLSAFHEPRYMGIALHNELLLSAAFHTARFVLVPSLHPDWTLLLFFFHTHSTVTTTLALIFIPKFWKLGAPPREEMVDEVCEDELDLQHSGSYLGSSIASAWSEHSLDPGDIRDELKKLYAQLEVHKTKEMAANNPHLPKKRGSSCQGLGRSFMRYLAEFPEALARQHSRDSGSPGHGSLPGSSRRRLLSSSLQEPEGTPALHKSRSTYDQRREQDPPLLDSLLRRKLAKKASRTESRESVEGPPALGFRSASAHNLTVGERLPRARPASLQKSLSVASSREKALLMASQAYLEETYRQAKEREERKKAKAAMASLVRRPSARRLERPRGAPLSAPPSPAKSSSVDSSHTSGRLHEEARRRLPHPPIRHQVSTPILALSGGLGEPRMLSPTSTLAPALLPALAPTPAPALAPVPVSPQSPNLLTYICPWENAELPAKQENVPQEGPSGPERGHHSPAPARARLWRALSVAVEKSRAGENEMDAEDAHHQREANDVDEDRPKIFPKSHSLKAPVQQGSMRSLGLAIKALTRSRSTYREKESVEESPEGQNSGTAGESMGAPSRSPRLGRPKAVSKQAALIPSDDKESLQNQQNAHTSRMLQVCQREGSREQEDRGRRMTQGLGERKAERAGKTGLAMLRQVSRDKNIKQSKETPVGWQELPKAGLQSLGSADHRVAEVCPWEVTESETRQPDSGNKAEICPWETSEGAPESRALRQDPGDSQKKRGEARGKSEPIDVVPMMRKKPERLVREQEAVCPWESADRGGLSPGSAPQDPGRIRDKSEAGDSVEARKVEKPGWEAAGPEAHTPDITKAEPCPWEASEGGEDGKPAQEAVKDLPQEKQKTRKATFWKEQKPGGDLESLCPWESTDFRGPSAVSIQAPGSSECSGSLGSGIAEVCLWEAGDAPAIQKAEICPWELDDNVMGQEMLSLGTGRESLQEKEKASRKGSFGEMGEQTVKAVQKLSQQQESVCPRESTVPGHSSPCLDNSSSKAGSQFLCNGGSRATQVCPQEDLRPEAQEATPAKTEICPWEVNERTREEWTSAQVPRGGESQKDKEKMPGKSEIEDVTAWEKPEGQIQKQEAVGPWESVDPGSFSPQPRPQDTERPQTLLQMSGSVGSKAADICPLDVEENLTAGKAEICPWEVGAGAGEERALGAEAIRKSPNDTGKVSADLGPRERAVTAPEKPQKPTPEWEVACPWGSVGPGACSQHPGTLDADGPKAGFQELDHMGCRPGEVCPWEAQEAATSEKAKICPWEVSEGTTGKGLDQKAGSESAEQREKALEKGRLTSLGEDVSKGMAKLCQQQETICIWENKDLRESPAQAPKISDLPSSMSSEVAEGHSLEATEKGDLRQDPKTGSFPEHITQEKAPAADTEEFTTEDGEKTSHELQSVCPWETTAPADSVSHLDRQRPDQPKASSQRLVSTGGRAADVCPWDVPDAGVYKSDSSAKAETCPWEVTERIPVKGVSRQDGKGDSQEEKGRAPEKSEPKGVPVQKKPEMADFRQQEAVCPWESQDGKGLSPQPAPDASDRSRGSSEAAGSVETRVAEVCLWEVVEAPSAKKAEICPWEAGGGAAEEGEQERESQGQGEMFLQKAGPGGTEEHFSKAAAKPREQEAVCPGEGTGSGGLLPQSGALDPELKVSPKEAGSMGSRMAELCQWEITDPEGNKIKGTMADICPGEETGVPSEESGLLALTATRREFFPTAPEKPLCLLVHGPLDHFFPESKIPCPKVSRPASTFTLEGVRELQGPSGLEPRTSLAPEPSLQEAESQSSSLTEDSGQVAFEAQYEEFTPPTVYPWDWE

SEQ ID NO: 3 - DNA, 21 bp, Artificial Sequence
  Synthetic oligonucleotide primer
ctcatacctacatccagaagc

SEQ ID NO: 4 - DNA, 21 bp, Artificial Sequence
  Synthetic oligonucleotide primer
catctccagagtagagataag

SEQ ID NO: 5 - DNA, 20 bp, Artificial Sequence
  Synthetic oligonucleotide primer
caggatctgtacccatgcag

SEQ ID NO: 6 - DNA, 20 bp, Artificial Sequence
  Synthetic oligonucleotide primer
cagttcccagacaagtcctg

SEQ ID NO: 7 - DNA, 21 bp, Artificial Sequence
  Synthetic oligonucleotide primer
gaggatgtggaatggtaccag

SEQ ID NO: 8 - DNA, 20 bp, Artificial Sequence
  Synthetic oligonucleotide primer
caggcatgcacatgttcgtg

SEQ ID NO: 9 - DNA, 20 bp, Artificial Sequence
  Synthetic oligonucleotide primer
gtgccataattccgagcatc

SEQ ID NO: 10 - DNA, 20 bp, Artificial Sequence
  Synthetic oligonucleotide primer
gtatctcacctctgccaatc

SEQ ID NO: 11 - DNA, 20 bp, Artificial Sequence
  Synthetic oligonucleotide primer
caggtagggtttccacagag

SEQ ID NO: 12 - DNA, 22 bp, Artificial Sequence
  Synthetic oligonucleotide primer
ctcatactcagaaggtgtagac

SEQ ID NO: 13 - DNA, 21 bp, Artificial Sequence
  Synthetic oligonucleotide primer
gatgtcaggatgctctagctg

SEQ ID NO: 14 - DNA, 21 bp, Artificial Sequence
  Synthetic oligonucleotide primer
cttgcatcatgctacacagtg

SEQ ID NO: 15 - DNA, 20 bp, Artificial Sequence
  Synthetic oligonucleotide primer
ctgagtgaccgttcacatag

SEQ ID NO: 16 - DNA, 21 bp, Artificial Sequence
  Synthetic oligonucleotide primer
cagagctcatagaccagcttg

SEQ ID NO: 17 - DNA, 21 bp, Artificial Sequence
  Synthetic oligonucleotide primer
gtggtgatggctctattgcag

SEQ ID NO: 18 - DNA, 21 bp, Artificial Sequence
  Synthetic oligonucleotide primer
caccattccatgagtgagctg

SEQ ID NO: 19 - DNA, 20 bp, Artificial Sequence
  Synthetic oligonucleotide primer
cagcaatggacagagaccag

SEQ ID NO: 20 - DNA, 21 bp, Artificial Sequence
  Synthetic oligonucleotide primer
gagaaccagaacaagaggaac

SEQ ID NO: 21 - DNA, 21 bp, Artificial Sequence
  Synthetic oligonucleotide primer
gttcctcttgttctggttctc

SEQ ID NO: 22 - DNA, 21 bp, Artificial Sequence
  Synthetic oligonucleotide primer
gtgaggagtactgttagagtg

SEQ ID NO: 23 - DNA, 20 bp, Artificial Sequence
  Synthetic oligonucleotide primer
ctcgtcctaagcgtatcagg

SEQ ID NO: 24 - DNA, 20 bp, Artificial Sequence
  Synthetic oligonucleotide primer
caagtcagctcctagcttag

SEQ ID NO: 25 - DNA, 21 bp, Artificial Sequence
  Synthetic oligonucleotide primer
gtgaagcgtcacaaagtaagg

SEQ ID NO: 26 - DNA, 21 bp, Artificial Sequence
  Synthetic oligonucleotide primer
cagtttaggccagtggagaag

SEQ ID NO: 27 - DNA, 22 bp, Artificial Sequence
  Synthetic oligonucleotide primer
ctggtctcacaggaacaaagag

SEQ ID NO: 28 - DNA, 19 bp, Artificial Sequence
  Synthetic oligonucleotide primer
gaattccgccaggtacctc

SEQ ID NO: 29 - DNA, 20 bp, Artificial Sequence
  Synthetic oligonucleotide primer
cacaggacgagctgaagaag

SEQ ID NO: 30 - DNA, 19 bp, Artificial Sequence
  Synthetic oligonucleotide primer
ctcctttgcttgccggtag

SEQ ID NO: 31 - DNA, 19 bp, Artificial Sequence
  Synthetic oligonucleotide primer
ctgcagaagtcgctcagtg

SEQ ID NO: 32 - DNA, 21 bp, Artificial Sequence
  Synthetic oligonucleotide primer
cagatgtaggtgagtaagttg

SEQ ID NO: 33 - DNA, 21 bp, Artificial Sequence
  Synthetic oligonucleotide primer
catcaggcaccaggtttctac

SEQ ID NO: 34 - DNA, 22 bp, Artificial Sequence
  Synthetic oligonucleotide primer
cacactctccttctctctgtag

SEQ ID NO: 35 - DNA, 21 bp, Artificial Sequence
  Synthetic oligonucleotide primer
gagaatgagatggacgcagag

SEQ ID NO: 36 - DNA, 20 bp, Artificial Sequence
  Synthetic oligonucleotide primer
gaaacttgcctcagcatggc

SEQ ID NO: 37 - DNA, 20 bp, Artificial Sequence
  Synthetic oligonucleotide primer
caggatgctccaagtctgtc

SEQ ID NO: 38 - DNA, 22 bp, Artificial Sequence
  Synthetic oligonucleotide primer
ctcagatttgtctctgattctg

SEQ ID NO: 39 - DNA, 22 bp, Artificial Sequence
  Synthetic oligonucleotide primer
catagatgtggttcccatgatg

SEQ ID NO: 40 - DNA, 21 bp, Artificial Sequence
  Synthetic oligonucleotide primer
catcacgttatcatccagctc

SEQ ID NO: 41 - DNA, 21 bp, Artificial Sequence
  Synthetic oligonucleotide primer
gtggcattgctgaagtgtgtc

SEQ ID NO: 42 - DNA, 20 bp, Artificial Sequence
  Synthetic oligonucleotide primer
gtcttgaggacgtggttgtg

SEQ ID NO: 43 - DNA, 21 bp, Artificial Sequence
  Synthetic oligonucleotide primer
gtgtccacaggaagatctcag

SEQ ID NO: 44 - DNA, 21 bp, Artificial Sequence
  Synthetic oligonucleotide primer
gtacctggatgctgagaacag

SEQ ID NO: 45 - DNA, 21 bp, Artificial Sequence
  Synthetic oligonucleotide primer
caggcaaggtttctgcagatc

SEQ ID NO: 46 - DNA, 20 bp, Artificial Sequence
  Synthetic oligonucleotide primer
gttctgggaaggaacctgtc

SEQ ID NO: 47 - DNA, 22 bp, Artificial Sequence
  Synthetic oligonucleotide primer
cagcagcatgagtagtgaagtg

SEQ ID NO: 48 - DNA, 22 bp, Artificial Sequence
  Synthetic oligonucleotide primer
ctgcttctgtcagaagcatctg

SEQ ID NO: 49 - DNA, 21 bp, Artificial Sequence
  Synthetic oligonucleotide primer
gatgttcctgatgcaggtgtg

SEQ ID NO: 50 - DNA, 21 bp, Artificial Sequence
  Synthetic oligonucleotide primer
ctgaaggaacatctctccttg

SEQ ID NO: 51 - DNA, 22 bp, Artificial Sequence
  Synthetic oligonucleotide primer
cagatgcttctgacagaagcag

SEQ ID NO: 52 - DNA, 20 bp, Artificial Sequence
  Synthetic oligonucleotide primer
caggacagatgtctgccatg

SEQ ID NO: 53 - DNA, 21 bp, Artificial Sequence
  Synthetic oligonucleotide primer
gacccagaactcaaagtcagc

SEQ ID NO: 54 - DNA, 21 bp, Artificial Sequence
  Synthetic oligonucleotide primer
caatcccaaggatagacagtg

SEQ ID NO: 55 - DNA, 21 bp, Artificial Sequence
  Synthetic oligonucleotide primer
gtgtcagagaactacaaggac

SEQ ID NO: 56 - DNA, 22 bp, Artificial Sequence
  Synthetic oligonucleotide primer
cttagaagtagagtgtccagtc

SEQ ID NO: 57 - DNA, 21 bp, Artificial Sequence
  Synthetic oligonucleotide primer
acgctggctctgatcttcatc

SEQ ID NO: 58 - DNA, 20 bp, Artificial Sequence
  Synthetic oligonucleotide primer
tgtgctcactccaggctgag

SEQ ID NO: 59 - DNA, 20 bp, Artificial Sequence
  Synthetic oligonucleotide primer
cgccaacacagtgctgtctg

SEQ ID NO: 60 - DNA, 20 bp, Artificial Sequence
  Synthetic oligonucleotide primer
ggagtacttgcgctcaggag

SEQ ID NO: 61 - DNA, 20 bp, Artificial Sequence
  Synthetic oligonucleotide primer
gagtgtcgcctacataaccg

SEQ ID NO: 62 - DNA, 20 bp, Artificial Sequence
  Synthetic oligonucleotide primer
cggttatgtaggcgacactc

SEQ ID NO: 63 - DNA, 20 bp, Artificial Sequence
  Synthetic oligonucleotide primer
gctgtactagctgcatggac

SEQ ID NO: 64 - DNA, 19 bp, Artificial Sequence
  Synthetic oligonucleotide primer
gcttcctggctgtatggac

SEQ ID NO: 65 - DNA, 19 bp, Artificial Sequence
  Synthetic oligonucleotide primer
gaggatgagctggatctgc

SEQ ID NO: 66 - DNA, 19 bp, Artificial Sequence
  Synthetic oligonucleotide primer
gtcgtcgtctgctgagcag

SEQ ID NO: 67 - DNA, 18 bp, Artificial Sequence
  Synthetic oligonucleotide primer
gtgctccgctgtctgctc

SEQ ID NO: 68 - DNA, 19 bp, Artificial Sequence
  Synthetic oligonucleotide primer
ccgtgctggcgaaaacgag

SEQ ID NO: 69 - DNA, 19 bp, Artificial Sequence
  Synthetic oligonucleotide primer
gaacgcaaagcagaacgcg

SEQ ID NO: 70 - DNA, 20 bp, Artificial Sequence
  Synthetic oligonucleotide primer
gtatgtccttgggaatccgc

SEQ ID NO: 71 - DNA, 18 bp, Artificial Sequence
  Synthetic oligonucleotide primer
gtttgcctgtgggaggcg

SEQ ID NO: 72 - DNA, 19 bp, Artificial Sequence
  Synthetic oligonucleotide primer
gaatggacctctgcgcagg

SEQ ID NO: 73 - DNA, 18 bp, Artificial Sequence
  Synthetic oligonucleotide primer
gcggttactgcaccggag

SEQ ID NO: 74 - DNA, 19 bp, Artificial Sequence
  Synthetic oligonucleotide primer
gcaccgaagatcagcgacc

SEQ ID NO: 75 - DNA, 20 bp, Artificial Sequence
  Synthetic oligonucleotide primer
gtgctgcagctgtttctgtc

SEQ ID NO: 76 - DNA, 20 bp, Artificial Sequence
  Synthetic oligonucleotide primer
aagaggaggagggtccagtc

SEQ ID NO: 77 - DNA, 20 bp, Artificial Sequence
  Synthetic oligonucleotide primer
cgccaacacagtgctgtctg

SEQ ID NO: 78 - DNA, 20 bp, Artificial Sequence
  Synthetic oligonucleotide primer
ggagtacttgcgctcaggag

SEQ ID NO: 79 - DNA, 23 bp, Artificial Sequence
  Synthetic oligonucleotide primer
gtgtgtccacaggaagatctcag

(19) United States
(12) Patent Application Publication
(10) Pub. No.: US 2015/0031569 A1
(43) Pub. Date: Jan. 29, 2015

(54) Mutations of the GPR179 Gene in Congenital Stationary Night Blindness

(71) Applicants: Centre National de la Recherche Scentifique, Paris (FR); Universite Pierre et Marie Curie (Paris 6), Paris (FR); Institut National de la Sante et de la Recherche Medicale (INSERM), Paris (FR); Assistance Publique - Hopitaux de Paris, Paris (FR)

(72) Inventors: Christina Zeitz, Paris (FR); Isabelle Audo, Paris (FR); Elise Orhan, Paris (FR); Kinga Jakowska, Paris (FR); Jose-Alain Sahel, Paris (FR)

(21) Appl. No.: 14/373,730

(22) PCT Filed: Jan. 23, 2013
(86) PCT No.: PCT/IB2013/050576

§ 371 Date: Jul. 22, 2014

(30) Foreign Application Priority Data
Jan. 23, 2012 (EP) 12305077.5

(51) Int. Cl. C12Q 1/68; C07K 14/72; G01N 33/50

(52) U.S. Cl. CPC C12Q 1/6883; G01N 33/5041; C12Q 1/6897; C07K 14/723; C12Q 2600/156; C12Q 2600/136; G01N 2333/726
USPC 506/9; 435/6.11; 435/7.21; 435/6.13; 536/23.5

(57) ABSTRACT

The present invention relates to an in vitro method for diagnosing a complete congenital stationary night blindness (cCSNB) in a subject, which method comprises determining the presence of an alteration in the GPR179 gene in a biological sample of said subject. Screening methods and therapeutic applications are further described.

The present invention relates to the identification of the GPR179 gene as a new gene involved in complete congenital stationary night blindness (cCSNB). Based on this identification, diagnostic methods, screening methods, and therapy of cCNSB, are proposed.

### BACKGROUND OF THE INVENTION

During mammalian retinal development a complex sequence of molecular events leads to the precise laminations and interconnections of the mature retina. In normal mature human retinas, rod and cone photoreceptors start the processing of vision, which proceeds through bipolar and ganglion cell retinal pathways to the brain. Hereditary disease can perturb these retinal pathways and cause either progressive degeneration or more stationary visual deficits. Congenital stationary night blindness (CSNB) is a group of retinopathies that fall into the latter category of a selective retinal pathway disturbance that manifest at birth.

CSNB comprises a group of genetically and clinically heterogeneous retinal disorders characterized by visual impairment under low light conditions. Frequent associated symptoms are myopia, nystagmus and strabismus. This disorder is due to a signal transmission defect from rod photoreceptors to adjacent bipolar cells in the retina. Two forms can be distinguished clinically, complete (c) or incomplete (ic) CSNB, depending on the affected signaling pathway.

The associated genes encode proteins, which are confined to the phototransduction cascade or are important in retinal signaling from photoreceptors to adjacent bipolar cells.

Most of the patients with mutations in these genes show a typical electrophysiological phenotype characterized by an electronegative waveform of the dark-adapted bright flash electroretinogram (ERG), in which the amplitude of the b-wave is smaller than that of the a-wave. This so-called Schubert-Bomschein type ERG response can be divided in two subtypes, incomplete (ic) CSNB (CSNB2A [MIM#300071], CSNB2B [MIM#610427]) and complete (c) CSNB (CSNB1A [MIM#310500], CSNB1B [MIM#257270] (Miyake et al, 1986) and CSNB1C [MIM#613216]).

icCSNB has been characterized by both a reduced rod b-wave and substantially reduced cone responses, due to both ON- and OFF-bipolar cell dysfunction, while the complete type is associated with a drastically reduced rod b-wave response due to ON-bipolar cell dysfunction, but largely normal cone b-wave amplitudes (Audo et al, 2008) icCSNB has been associated with mutations in CACNA1F [MIM#300110], CABP4 [MIM#608965] and CACNA2D4 [MIM#608171], while cCSNB has been associated with mutations in NYX [MIM#300278], GRM6 [MIM#604096] and TRPM1 [MIM#603576]. More than 280 mutations have been identified in these genes using direct sequencing of candidate genes or microarray analysis (Zeitz et al, 2009).

Nonetheless, up to this point, no single biomarker is sufficiently specific to provide adequate clinical utility for the diagnosis of complete CSNB in an individual subject. Therefore, there is a need for identifying other mutations responsible for complete CSNB. So far mutations in several genes leading to CSNB have been identified through a candidate gene approach by comparing the human phenotype to similar phenotypes observed in knock-out or naturally occurring animal models (Dryja et al, 2005, Zeitz et al, 2005 and 2006, Wycisk et al, 2006, Audo et al, 2009, Li et al, 2009, Van Genderen et al, 2009, Nakamura et al, 2010). The bottleneck of this approach is the size of a cohort and the identification of the “right” patient harboring the mutation in such a candidate gene.

The identification of the genes which are causative of complete CSNB may allow for development of differential diagnostic tests for this disorder and risk assessment in affected families. As well, identification of such genes will provide information as to the basic defect in this retinal condition, which could lead to effective methods for treatment or cure of the disorder.

### SUMMARY OF THE INVENTION

The present invention provides a method, preferably an in vitro method, for diagnosing an autosomal recessive complete congenital stationary night blindness (cCSNB) in a subject, which method comprises determining the presence of an alteration in the GPR179 gene in a biological sample of said subject.

The invention further provides a method, preferably an in vitro method, for determining the risk for a subject to transmit an autosomal recessive complete congenital stationary night blindness (cCSNB) to his/her progeny, which method comprises determining the presence of an alteration in the GPR179 gene in a biological sample of said subject.

Preferably, for determining the risk for a subject to transmit an autosomal recessive complete congenital stationary night blindness (cCSNB) to his/her progeny, a suitable biological sample may be obtained from germline cells.

Preferably the alteration is a mutation, deletion, or addition of one or more nucleotides in at least one exon of the GPR179 gene, or in a splicing donor or acceptor site.

The invention further provides methods, preferably in vitro methods, for selecting compounds as candidate medicaments for treating autosomal recessive cCSNB.

It is further described a method for treating an autosomal recessive cCSNB in a subject, which method comprises administering the subject with a nucleic acid encoding GPR179 protein or a ligand, preferably an agonist, of the GPR179 protein.

### LEGEND TO THE FIGURES

FIG. 1 shows GPR179 mutations in cCSNB.

a) GPR179 gene structure containing 11 coding exons (Ref Seq NM_{—}001004334.2). Different mutations identified in cCSNB patients are depicted.

b) The specific domains for GPR179 were estimated by a prediction program (UniProtKB/Swiss-Prot, www.uniprot.org).

FIG. 2 shows a 3-dimensional (3-D) model of the trans-membrane region of GPR179 A) 3D homology model based on the 3D model of the wild-type squid rhodopsin (2ZIY). Wild-type Gly455 and His603 residues are indicated in green and the mutated Asp455 and Tyr603 in orange. B) Superimposition of 3-D models of the wild-type and the mutation p.Gly455ASP (aspartate in orange).

FIG. 3 is a diagram that shows genes underlying CSNB. Different forms of CSNB in human are classified according to their electroretinographic feature, mode of inheritance, the clinical phenotype and mutated genes. Patients discussed herein, show a complete Schubert-Bornschein type of ERG. Abbreviations: cCSNB=complete CSNB; icCSNB=incomplete CSNB; ar=autosomal recessive, ad=autosomal dominant. Genes are indicated in italics and underlined. Chromosomal location is given between brackets. The phenotype of patients with mutations in icCSNB is more variable and can even lead to progressive cone or cone-rod dystrophy 1.

FIG. 4 shows the cellular localization of wild-type and mutated GPR179 in COS-1 cells. Extracellular (green, left-hand column 1) and intracellular (red, middle column 2) staining was performed on COS-1 cells expressing normal (line 1) and p.Asp126His (line2), p.Tyr220Cys (line3), pGly455Asp (line4), and p.His603Tyr (line5) mutated GPR179. An overlay of theses stainings and DAPI stained nuclei are depicted on column 3 (right-hand column). Scale bar=20 μm.

FIG. 5 shows that the c.1784+1G>A GPR179 mutation interferes with splicing. A: Schematic drawing of minigenes used to analyze GPR179 (NM_{—}001004334.2) splicing. We compared splicing of GPR179 control (mini-wt) and mutated (mini-mut) alleles and cloned an amplicon containing intron 6 through 9 into the multiple cloning site of a vector (pCRII-TOPO). The horizontal arrows show binding sites of GPR179_EX7F and GPR179_EX9R oligonucleotides used for patient gDNA PCR and RT_GPR179_EX7F and RT_GPR179_EX9R primers used for RT-PCR analysis. The mutation c.1784+1G>A and the alternative c.1784+63 splice site are depicted by vertical arrows. B: Representative RT-PCR analyses of transfected COS 1 cells revealed two major transcripts (286 by and 426 bp) for normal (wt) and mutated (mut) constructs (286 by and 488 bp) respectively. C: Schematic drawing of different splice transcripts identified by sequencing. The mini-wt 426 by transcript includes complete exons 7, 8 and 9, whereas the mini-wt 286 by isoform skips exon 8. The mini-mut 286 by isoform is the same than the mini-wt 286 by and the mini-mut 488 by isoform include exons 7, 8 and a part of intron 8 and exon 9. D: Quantitative RT-PCR showed a significant increase of exon 8 skipping in mini-mut compared to mini-wt (n=5, *** representing the p value=0.005).

### DETAILED DESCRIPTION OF THE INVENTION

Prevalence studies determined that CACNA1F, NYX and TRPM1 mutations leading to incomplete and complete CSNB occur more frequently (unpublished data). Genotyping studies of a CSNB cohort comprising 160 patients revealed that in about 13% of cases mutations in known genes underlying CSNB were not identified. This was a strong indication that mutations in other genes remained to be discovered, or that mutations in non-screened regions, i.e. regulatory elements and introns, may be involved.

On this basis, the inventors have worked to identify the missing mutations in this CSNB cohort and have found a new gene involved in CSNB, namely the GPR179 gene.

### DEFINITIONS

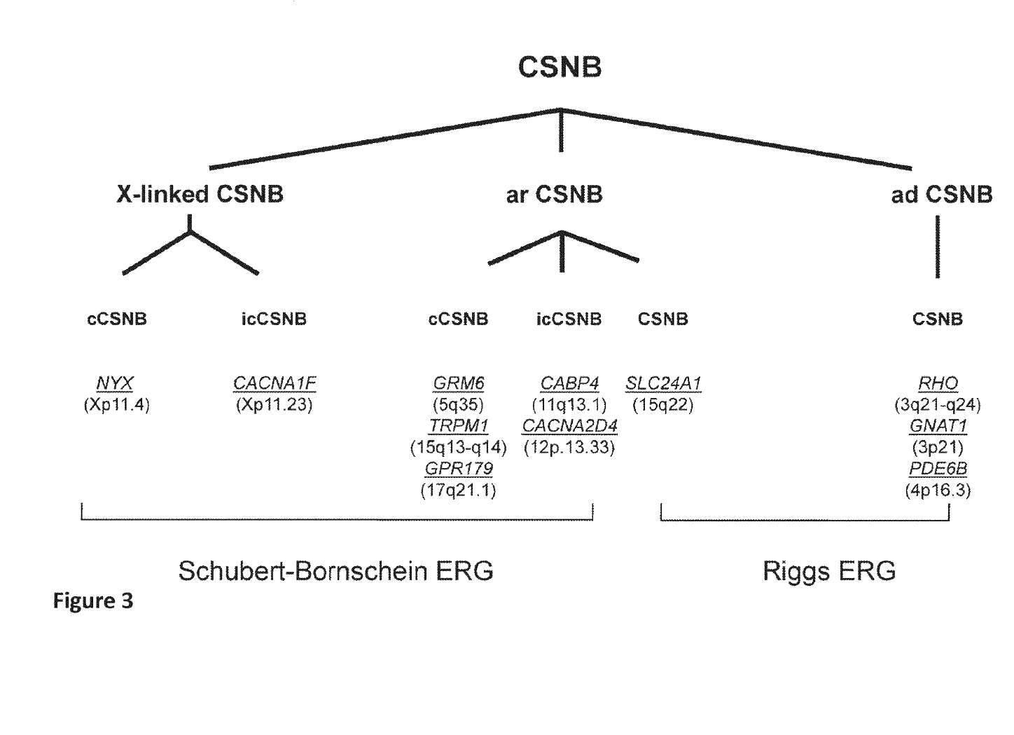

Unless otherwise specified, Congenital stationary night blindness means any form of CSNB, including complete or incomplete forms, regardless of the inheritance mode. Indeed CSNB can be inherited by an autosomal dominant, autosomal recessive, or X-linked mode. The method of the invention preferably allows a diagnosis of complete CSNB, more particularly autosomal recessive cCSNB (see FIG. 3). Especially it allows for a differential diagnosis of cCSNB with all forms of night blindness associated with the early stages of retinitis pigmentosa.

As used in the present application, the term “GPR179 gene” designates the G protein-coupled receptor 179. This gene is described in Zody et al, Nature 440 (7087), 1045-1049 (2006) and Bjarnadottir, et al, Gene 362, 70-84 (2005).

Preferably it refers to the human GPR179 gene, however orthologous genes are encompassed when the subject is a non-human mammal. The sequence of the human cDNA starting from the translation-initiation codon ATG is shown as SEQ ID NO: 1 (NCBI Reference Sequence: NM_{—}001004334.2), the sequence of the human protein is shown as SEQ ID NO:2. Naturally-occurring variants due to allelic variations between individuals (e.g., polymorphisms) are encompassed in the present invention.

Within the context of this invention, the GPR179 gene designates all GPR179 sequences or products in a cell or organism, including GPR179 coding sequences, GPR179 non-coding sequences (e.g., introns), GPR179 regulatory sequences controlling transcription and/or translation (e.g., promoter, enhancer, terminator, etc.), as well as all corresponding expression products, such as GPR179 RNAs (e.g., mRNAs) and GPR179 polypeptide (e.g., a pre-protein and a mature protein).

The term “gene” shall be construed to include any type of coding nucleic acid, including genomic DNA (gDNA), complementary DNA (cDNA), synthetic or semi-synthetic DNA, as well as any form of corresponding RNA.

A GPR179 “protein” or “polypeptide” designates any protein or polypeptide encoded by a GPR179 gene as disclosed above. The term “polypeptide” refers to any molecule comprising a stretch of amino acids. This term includes molecules of various lengths, such as peptides and proteins. The polypeptide may be modified, such as by glycosylations and/or acetylations and/or chemical reactions or coupling, and may contain one or several non-natural or synthetic amino acids. A specific example of a GPR179 polypeptide comprises all or part of SEQ ID NO: 2. At the protein level, it is noteworthy that the major differences between GPR179 and the closely related GPR158 family rely on the absence of the calcium-binding EGF-like domain at the N-terminal part and a reduced number of CPWE motifs (up to 3) in all members of the GPR158 relatives. Interestingly, three other molecules, the regulator of G-protein signaling 9 (RGS9 [MIM:604067]), the retinal rod rhodopsin-sensitive cGMP 3,5-cyclic phosphodiesterase subunit gamma(PDE6G [MIM18073]) and the retinal cone rhodopsin-sensitive cGMP 3,5-cyclic phosphodiesterase subunit gamma (PDE6H [MIM:601190]) share the same protein motif CPWE. These molecules have been implicated in the inhibition of the G-protein or amplification of the signal in the phototransduction cascade. Mutations in those genes lead to different retinal disorders including bradyopsia [MIM: 608415] (Nishiguchi et al, 2004), rod-cone dystrophy [MIM: 613582] (Dvir et al, 2010) and cone dystrophy [MIM:610024].

A “subject” is preferably a mammal. The mammal can be a human, non-human primate, mouse, rat, dog, cat, horse, or cow, but are not limited to these examples. A subject can be male or female, of any age, including a foetus. A subject may be any individual that shows symptoms of CSNB, or a subject that has been diagnosed with a CSNB. A subject can also be one who has not been previously diagnosed as having CSNB. For example, a subject can be one who exhibits one or more risk factors for CSNB, or a subject who is asymptomatic for CSNB. A subject can also be one who is at risk of developing CSNB, or at risk of transmitting the disease to its progeny. In a particular embodiment, the subject may be related to an individual, e.g. a sibling or a parent, with a CSNB.

Within the context of the present invention, the term “diagnosis” includes the detection or confirmation, at various stages, including early, pre-symptomatic stages, and late stages, in adults or children. Prenatal diagnostics may also be contemplated.

A “biological sample” may be of any type. Examples of such samples include fluids, tissues, cell samples, organs, biopsies, etc. In particular, especially when DNA has to be extracted, the biological sample may be a fluid sample, e.g. blood or urine, or it may be fibroblasts or keratinocytes for instance. Preferred biological samples are whole blood, serum, plasma or urine. In the prospect of in vitro fertilization, or sperm selection, or for determining the risk for a subject to transmit an autosomal recessive complete congenital stationary night blindness (cCSNB) to his/her progeny, the biological sample may also be male and/or female gametes. Typically, in the case where the subject is a foetus, the sample may be an amniosynthesis sample.

Diagnosis

The invention now provides diagnosis methods based on a monitoring of the GPR179 gene in a subject.

The present invention provides a method, preferably an in vitro method, for diagnosing an autosomal recessive complete congenital stationary night blindness (cCSNB) in a subject, which method comprises determining the presence of an alteration in the GPR179 gene in a biological sample of said subject.

Since the disease is recessive, the two alleles of the gene must carry the mutation to develop the disease and therefore must be tested.

The invention is also useful to test whether a subject carries a risk-allele for an autosomal recessive complete congenital stationary night blindness, and therefore might transmit the disease to his or her progeny.

The present invention thus further provides a method, preferably an in vitro method, for determining the risk for a subject to transmit an autosomal recessive complete congenital stationary night blindness (cCSNB) to his/her progeny, which method comprises determining the presence of an alteration in the GPR179 gene in a biological sample of said subject.

Optionally, said methods comprise a preliminary step of providing a sample from a subject.

Preferably, the presence of an alteration in the GPR179 gene in said sample is detected through the genotyping of a sample.

The method may further comprise determining the presence of an alteration in at least one of the following genes: NYX, CACNA1F, GRM6, TRPM1, CABP4, CACNA2D4, SLC24A1, RHO, GNAT1 and PDE6B.

In particular mutations in NYX, GRM6, and TRPM1 have been shown to be implicated in cCSNB (FIG. 3). Dryja et al, 2005, Zeitz et al, 2005, Audo et al, 2009, Li et al, 2009, Van Genderen et al, 2009, Nakamura et al, 2010, Bech-Hansen et al, 2000, Pusch et al, 2000).

These genes code for nyctalopin, metabotropic glutamate receptor 6, and transient receptor potential cation melastatin 1 channel. All but GPR179 localize postsynaptically to the photoreceptors in the retina in ON-bipolar cells (Morgans et al, 2006).

The alteration may be determined at the level of the GPR179 gDNA, RNA or polypeptide. Optionally, the detection is performed by sequencing all or part of the GPR179 gene or by selective hybridization or amplification of all or part of the GPR179 gene. More preferably a GPR179 gene specific amplification is carried out before the alteration identification step.

An alteration in the GPR179 gene locus may be any form of mutation(s), deletion(s), rearrangement(s) and/or insertions in the coding and/or non-coding region of the locus, alone or in various combination(s). Mutations more specifically include point mutations. Deletions may encompass any region of two or more residues in a coding or non-coding portion of the gene locus, such as from two residues up to the entire gene or locus. Typical deletions affect smaller regions, such as domains (introns) or repeated sequences or fragments of less than about 50 consecutive base pairs, although larger deletions may occur as well. Insertions may encompass the addition of one or several residues in a coding or non-coding portion of the gene locus. Insertions may typically comprise an addition of between 1 and 50 base pairs in the gene locus. Rearrangement includes inversion of sequences. The GPR179 gene locus alteration may result in the creation of stop codons, frameshift mutations, amino acid substitutions, particular RNA splicing or processing, product instability, truncated polypeptide production, etc. The alteration may result in the production of a GPR179 polypeptide with altered function, stability, targeting or structure.

The alteration may also cause a reduction in protein expression.

Preferably the alteration is a mutation, deletion, or addition of one or more nucleotides in at least one exon of the GPR179 gene, or in a splicing donor or acceptor site.

Preferably the subject is homozygote for the alteration. Or the subject may be compound heterozygote for the alteration(s), which means that a copy of the GPR179 gene may carry one alteration, while the other copy carries another alteration in the GPR179 gene.

More particularly, the alteration to detect may be selected from the group consisting of
i. a deletion of nucleotide C at position 278 on SEQ ID NO:1;
ii. a substitution of nucleotide G into C at position 376 on SEQ ID NO:1;
iii. a deletion of nucleotides 479 to 501 on SEQ ID NO:1;
iv. a substitution of nucleotide C into T at position 598 on SEQ ID NO:1;
v. a deletion of nucleotide C at position 984 on SEQ ID NO:1;
vi. a substitution of nucleotide G into A at position 1364 on SEQ ID NO:1;
vii. a substitution of nucleotide G into A at position 1784+1 on SEQ ID NO:1;
viii. a substitution of nucleotide C into T at position 1807 on SEQ ID NO:1.

Alterations identified by the inventors are shown in Table 1.

Whole exome sequencing in cCSNB cases lacking mutations in the known genes led to the identification of a homozygous missense mutation (c.1807C>T, p.His603Tyr) in one consanguineous autosomal recessive cCSNB family and a homozygous frameshift mutation (c.278delC, p.Pro93Glnfs*57) in a simplex male cCSNB patient in GPR179. Additional screening using Sanger sequencing of 40 patients identified 3 other cCSNB cases harboring additional allelic mutations in GPR179 (p.Asp126His, p.Ser329Leufs*4, p.Leu160Profs*38. P.Gly455Asp, p.Arg200*, c.1784+1G>A).

Others studies reported two families with p.Leu63Serfs*12, p.Ser329Leufs*4 and p.Tyr220Cys mutations in the same gene (Peachey et al., 2012).

Others mutations which may be detected may be a substitution of nucleotide A into G at position 659 on SEQ ID NO:1; or a deletion of a nucleotide at position 187 on SEQ ID NO:1.

Preferably the presence of an alteration in the GPR179 gene may be determined by sequencing, selective hybridization and/or selective amplification, as described in greater details below.

In another embodiment, the presence of an alteration in the GPR179 gene is determined by detecting a mutation in the amino acid sequence of the protein encoded by said gene.

More particularly, a deletion of nucleotide C at position 278 on SEQ ID NO:1 results in a frameshift in expression, whereby the protein is either truncated (at position 93 on SEQ ID NO:2) or not expressed.

A substitution of nucleotide G into C at position 376 on SEQ ID NO:1 results in a substitution of amino acid Asp into His at position 126 of SEQ ID NO:2.

A deletion of nucleotides 479 to 501 on SEQ ID NO:1 results in a frameshift of the protein, whereby the protein is either truncated (at position 160 on SEQ ID NO:2) or not expressed.

A substitution of nucleotide C into T at position 598 on SEQ ID NO:1; results in the creation of a stop codon, whereby the protein is truncated at amino acid 200 on SEQ ID NO:2.

A deletion of nucleotide C at position 984 on SEQ ID NO:1 results in a frameshift of the protein, whereby the protein is either truncated (at position 329 on SEQ ID NO:2) or not expressed.

A substitution of nucleotide G into A at position 1364 on SEQ ID NO:1 results in a substitution of amino acid Gly into Asp at position 455 of SEQ ID NO:2.

A substitution of nucleotide G into A at position 1784+1 on SEQ ID NO:1 results in a splicing defect, whereby either a frameshift of the protein is generated leading to altered protein expression or no expression, or an exon is skipped leading again to altered protein expression or no expression.

A substitution of nucleotide C into T at position 1807 on SEQ ID NO:1 results in a substitution of amino acid His into Tyr at position 603 of SEQ ID NO:2.

A substitution of nucleotide A into G at position 659 on SEQ ID NO:1 results in a substitution of amino acid Tyr into Cys at position 220 of SEQ ID NO:2.

A deletion of nucleotide at position 187 on SEQ ID NO:1 results in a frameshift of the protein, whereby the protein is either truncated (at position 63 on SEQ ID NO:2) or not expressed.

In another embodiment, the presence of an alteration in the GPR179 gene is determined by determining the level of expression of the GPR179 protein in a biological sample of the subject, wherein an absence of expression or a decreased level of expression of the GPR179 protein with respect to a healthy control is indicative of a cCSNB. In that case the biological sample is preferably fibroblasts.

More particularly, the method may comprise detecting the presence of an altered GPR179 RNA expression. Altered RNA expression includes the presence of an altered RNA sequence, the presence of an altered RNA splicing or processing, the presence of an altered quantity of RNA, etc. These may be detected by various techniques known in the art, including by sequencing all or part of the GPR179 RNA or by selective hybridization or selective amplification of all or part of said RNA, for instance.

In a further variant, the method may comprise detecting the presence of an altered GPR179 polypeptide expression. Altered GPR179 polypeptide expression includes the presence of an altered polypeptide sequence, the presence of an altered quantity of GPR179 polypeptide, the presence of an altered tissue distribution, etc. These may be detected by various techniques known in the art, including by sequencing and/or binding to specific ligands (such as antibodies), for instance.

In a preferred embodiment, once an alteration in the genomic sequence of the GPR179 gene of a patient has been identified, cell hosts overexpressing said altered sequence may be used to determine whether said alteration has a functional impact on the protein.

Various techniques known in the art may be used to detect or quantify altered GPR179 gene or RNA expression or sequence, including sequencing, hybridization, amplification and/or binding to specific ligands (such as antibodies). Other suitable methods include allele-specific oligonucleotide (ASO), allele-specific amplification, Southern blot (for DNAs), Northern blot (for RNAs), single-stranded conformation analysis (SSCA), PFGE, fluorescent in situ hybridization (FISH), gel migration, clamped denaturing gel electrophoresis, heteroduplex analysis, RNase protection, chemical mismatch cleavage, ELISA, radio-immunoassays (RIA) and immuno-enzymatic assays (IEMA).

Some of these approaches (e.g., SSCA and CGGE) are based on a change in electrophoretic mobility of the nucleic acids, as a result of the presence of an altered sequence. According to these techniques, the altered sequence is visualized by a shift in mobility on gels. The fragments may then be sequenced to confirm the alteration.

Some others are based on specific hybridization between nucleic acids from the subject and a probe specific for wild type or altered GPR179 gene or RNA. The probe may be in suspension or immobilized on a substrate. The probe is typically labeled to facilitate detection of hybrids.

Some of these approaches are particularly suited for assessing a polypeptide sequence or expression level, such as Northern blot, ELISA and RIA. These latter require the use of a ligand specific for the polypeptide, more preferably of a specific antibody.

In a particular, preferred, embodiment, the method comprises detecting the presence of an altered GPR179 gene expression profile in a sample from the subject. As indicated above, this can be accomplished more preferably by sequencing, selective hybridization and/or selective amplification of nucleic acids present in said sample.

Sequencing

Sequencing can be carried out using techniques well known in the art, using automatic sequencers. The sequencing may be performed on the complete GPR179 gene or, more preferably, on specific domains thereof, typically those known or suspected to carry deleterious mutations or other alterations, e.g. in particular exons.

Amplification

Amplification is based on the formation of specific hybrids between complementary nucleic acid sequences that serve to initiate nucleic acid reproduction.

Amplification may be performed according to various techniques known in the art, such as by polymerase chain reaction (PCR), ligase chain reaction (LCR), strand displacement amplification (SDA) and nucleic acid sequence based amplification (NASBA). These techniques can be performed using commercially available reagents and protocols.

Preferred techniques use allele-specific PCR or PCR-SSCP. Amplification usually requires the use of specific nucleic acid primers, to initiate the reaction.

Nucleic acid primers useful for amplifying sequences from the GPR179 gene are able to specifically hybridize with a portion of the GPR179 gene that flank a target region of said gene.

Primers may be designed based on the sequence of SEQ ID No 1 or on the genomic sequence of GPR179. Typical primers of this invention are single-stranded nucleic acid molecules of about 5 to 60 nucleotides in length, more preferably of about 8 to about 25 nucleotides in length. The sequence can be derived directly from the sequence of the GPR179 gene locus. Perfect complementarity is preferred, to ensure high specificity. However, certain mismatch may be tolerated.

In a particular embodiment, useful primers are shown in Table 2.

Selective Hybridization

Hybridization detection methods are based on the formation of specific hybrids between complementary nucleic acid sequences that serve to detect nucleic acid sequence alteration(s).

A particular detection technique involves the use of a nucleic acid probe specific for wild type or altered GPR179 gene or RNA, followed by the detection of the presence of a hybrid. The probe may be in suspension or immobilized on a substrate or support (as in nucleic acid array or chips technologies). The probe is typically labeled to facilitate detection of hybrids.

In this regard, a particular embodiment of this invention comprises contacting the sample from the subject with a nucleic acid probe specific for an altered GPR179 gene, and assessing the formation of a hybrid. In a particular, preferred embodiment, the method comprises contacting simultaneously the sample with a set of probes that are specific, respectively, for wild type GPR179 gene locus and for various altered forms thereof. In this embodiment, it is possible to detect directly the presence of various forms of alterations in the GPR179 gene in the sample. Also, various samples from various subjects may be treated in parallel.

Within the context of this invention, a probe refers to a polynucleotide sequence which is complementary to and capable of specific hybridization with a (target portion of a) GPR179 gene or RNA, and which is suitable for detecting polynucleotide polymorphisms associated with GPR179 alleles which predispose to or are associated with CSNB. Probes are preferably perfectly complementary to the GPR179 gene, RNA, or target portion thereof

Probes typically comprise single-stranded nucleic acids of between 8 to 1000 nucleotides in length, for instance of between 10 and 800, more preferably of between 15 and 700, typically of between 20 and 500. It should be understood that longer probes may be used as well. A preferred probe of this invention is a single stranded nucleic acid molecule of between 8 to 500 nucleotides in length, which can specifically hybridize to a region of a GPR179 gene or RNA that carries an alteration.

A specific embodiment of this invention is a nucleic acid probe specific for an altered (e.g., a mutated) GPR179 gene or RNA, i.e., a nucleic acid probe that specifically hybridizes to said altered GPR179 gene or RNA and essentially does not hybridize to a GPR179 gene or RNA lacking said alteration. Specificity indicates that hybridization to the target sequence generates a specific signal which can be distinguished from the signal generated through non-specific hybridization. Perfectly complementary sequences are preferred to design probes according to this invention. It should be understood, however, that a certain degree of mismatch may be tolerated, as long as the specific signal may be distinguished from non-specific hybridization.

The sequence of the probes can be derived from the sequences of the GPR179 gene and RNA as provided in the present application. Nucleotide substitutions may be performed, as well as chemical modifications of the probe. Such chemical modifications may be accomplished to increase the stability of hybrids (e.g., intercalating groups) or to label the probe. Typical examples of labels include, without limitation, radioactivity, fluorescence, luminescence, enzymatic labeling, etc.

Specific Ligand Binding

As indicated above, alteration in the GPR179 gene locus may also be detected by screening for alteration(s) in GPR179 polypeptide sequence or expression levels in vitro. In this regard, a specific embodiment of this invention comprises contacting the sample with a ligand specific for a GPR179 polypeptide and determining the formation of a complex.

Different types of ligands may be used, such as specific antibodies. In a specific embodiment, the sample is contacted with an antibody specific for a GPR179 polypeptide and the formation of an immune complex is determined. Various methods for detecting an immune complex can be used, such as immunolocalisation, ELISA, radioimmunoassay (RIA) and immuno-enzymatic assays (IEMA).

Within the context of this invention, an antibody designates a polyclonal antibody, a monoclonal antibody, as well as fragments or derivatives thereof having substantially the same antigen specificity. Fragments include Fab, Fab′2, CDR regions, etc. Derivatives include single-chain antibodies, humanized antibodies, poly-functional antibodies, etc.

An antibody specific for a GPR179 polypeptide designates an antibody that selectively binds a GPR179 polypeptide, namely, an antibody raised against a GPR179 polypeptide or an epitope-containing fragment thereoflthough non-specific binding towards other antigens may occur, binding to the target GPR179 polypeptide occurs with a higher affinity and can be reliably discriminated from non-specific binding.

In a specific embodiment, the method comprises contacting a biological sample from the subject, or a protein mimicking the patient from overexpressing cells lines with (a support coated with) an antibody specific for an altered form of a GPR179 polypeptide, and determining the presence of an immune complex. In a particular embodiment, the sample may be contacted simultaneously, or in parallel, or sequentially, with various (supports coated with) antibodies specific for different forms of a GPR179 polypeptide, such as a wild type and various altered forms thereof.

In order to carry out the methods of the invention, one can employ diagnostic kits comprising products and reagents for detecting the presence of an alteration in the GPR179 gene or polypeptide, in the GPR179 gene or polypeptide expression, and/or in GPR179 activity. Said diagnostic kit comprises any primer, any pair of primers, any nucleic acid probe and/or any ligand, preferably antibody, described in the present invention. Said diagnostic kit can further comprise reagents and/or protocols for performing a hybridization, amplification or antigen-antibody immune reaction.

The diagnosis methods can be performed in vitro, ex vivo or in vivo, preferably in vitro or ex vivo. They use a sample from the subject, to assess the status of the GPR179 gene. The sample may be any biological sample derived from a subject, which contains nucleic acids or polypeptides. Examples of such samples include fluids, tissues, cell samples, organs, biopsies, etc. Most preferred samples are DNA from blood or fibroblasts, plasma, saliva, urine, seminal fluid, etc. The sample may be collected according to conventional techniques and used directly for diagnosis or stored. The sample may be treated prior to performing the method, in order to render or improve availability of nucleic acids or polypeptides for testing. Treatments include, for instance, lysis (e.g., mechanical, physical, chemical, etc.), centrifugation, etc. Also, the nucleic acids and/or polypeptides may be pre-purified or enriched by conventional techniques, and/or reduced in complexity. Nucleic acids and polypeptides may also be treated with enzymes or other chemical or physical treatments to produce fragments thereof. Considering the high sensitivity of the claimed methods, very few amounts of sample are sufficient to perform the assay.

As indicated, the sample is preferably contacted with reagents such as probes, primers or ligands in order to assess the presence of an altered GPR179 gene. Contacting may be performed in any suitable device, such as a plate, tube, well, glass, etc. In specific embodiments, the contacting is performed on a substrate coated with the reagent, such as a nucleic acid array or a specific ligand array. The substrate may be a solid or semi-solid substrate such as any support comprising glass, plastic, nylon, paper, metal, polymers and the like. The substrate may be of various forms and sizes, such as a slide, a membrane, a bead, a column, a gel, etc. The contacting may be made under any condition suitable for a complex to be formed between the reagent and the nucleic acids or polypeptides of the sample.

The finding of an altered GPR179 polypeptide, RNA or DNA in the sample is indicative of the presence of an altered GPR179 gene in the subject, which can be correlated to the presence of an autosomal recessive cCSNB. The determination of the presence of an altered GPR179 gene in a subject also allows the design of appropriate therapeutic intervention, which is more effective and customized.

Drug Screening

The present invention also provides novel targets and methods for the screening of drug candidates or leads. The methods include binding assays and/or functional assays, and may be performed in vivo, in vitro, in cell systems, in animals, etc.

In a first aspect, it is provided a method, preferably an in vitro method, of selecting compounds as candidate medicaments for treating autosomal recessive cCSNB, said method comprising contacting a test compound with a GPR179 protein or gene or a fragment thereof and determining the ability of said test compound to bind the GPR179 protein or gene or a fragment thereof

Binding to said gene or polypeptide provides an indication as to the ability of the compound to modulate the activity of said target, and thus to affect a pathway leading to cCSNB.

The determination of binding may be performed by various techniques, such as by labeling of the test compound, by competition with a labeled reference ligand, etc.

In a second aspect, it is provided a method, preferably an in vitro method, of selecting compounds as candidate medicaments for treating autosomal recessive cCSNB, said method comprising contacting a test compound with a recombinant host cell expressing a GPR179 protein, and determining the ability of said test compound to bind said GPR179 protein and to modulate the activity of the GPR179 protein.

In a third aspect, it is provided a method, preferably an in vitro method, of selecting compounds as candidate medicaments for treating autosomal recessive cCSNB, said method comprising contacting a test compound with a GPR179 gene and determining the ability of said test compound to modulate (preferably stimulate) the expression of said gene.

In a fourth aspect, it is provided a method, preferably an in vitro method, of selecting compounds as candidate medicaments for treating autosomal recessive cCSNB, said method comprising contacting a test compound with a recombinant host cell comprising a reporter construct, said reporter construct comprising a reporter gene under the control of a GPR179 gene promoter, and selecting the test compounds that modulate (preferably stimulates) expression of the reporter gene.

More particularly, said GPR179 protein or gene or a fragment thereof is an altered or mutated GPR179 protein or gene or a fragment thereof comprising the alteration or mutation.

The above screening assays may be performed in any suitable device, such as plates, tubes, dishes, flasks, etc. Typically, the assay is performed in multi-wells plates. Several test compounds can be assayed in parallel. Furthermore, the test compound may be of various origin, nature and composition. It may be any organic or inorganic substance, such as a lipid, peptide, polypeptide, nucleic acid, small molecule, etc., in isolated or in mixture with other substances. The compounds may be all or part of a combinatorial library of products, for instance.

Pharmaceutical Compositions, Therapy

The present invention identifies mutations in the GPR179 gene that are involved in cCSNB.

The invention thus provides a novel target of therapeutic intervention. Various approaches can be contemplated to restore or modulate the GPR179 activity or function in a subject, particularly those carrying an altered GPR179 gene. Supplying wild-type function to such subjects is expected to suppress phenotypic expression of cCSNB in a pathological cell or organism. The supply of such function can be accomplished through gene or protein therapy, or by administering compounds that modulate or mimic GPR179 polypeptide activity (e.g., agonists as identified in the above screening assays).

Other molecules with GPR179 activity (e.g., peptides, drugs, GPR179 agonists, or organic compounds) may also be used to restore functional GPR179 activity in a subject or to suppress the deleterious phenotype in a cell.

Restoration of functional GPR179 gene function in a cell may be used to alleviate symptoms of cCNSB or to cure the disease.

A further object of this invention is a pharmaceutical composition comprising (i) a GPR179 polypeptide or a fragment thereof, a nucleic acid encoding a GPR179 polypeptide or a fragment thereof, a vector or a recombinant host cell as described above and (ii) a pharmaceutically acceptable carrier or vehicle.

The invention also relates to a method of treating autosomal recessive cCSNB, in a subject, the method comprising administering to said subject a functional (e.g., wild-type) GPR179 polypeptide or a nucleic acid encoding the same.

Another embodiment of this invention resides in a method of treating autosomal recessive cCSNB in a subject, the method comprising administering to said subject a compound that modulates, preferably that activates or mimics, expression or activity of a GPR179 gene or protein according to the present invention. In a particular embodiment of the method, the compound is a ligand, preferably an agonist of the GPR179 protein.

Such medicament may be administered by any route, preferably by the ocular route.

The wild-type GPR179 gene or a functional part thereof may be introduced into the cells of the subject in need thereof using a vector as described above. The vector may be a viral vector or a plasmid. The gene may also be introduced as naked DNA.

In a particular embodiment, viral vectors (e.g., replication defective retroviruses, adenoviruses and adeno-associated viruses (AAV)) are employed. In a preferred embodiment, the expression vector is an AAV vector. Adeno-associated viral (AAV) vectors have become powerful gene delivery tools for the treatment of retinal degeneration. AAV vectors possess a number of features that render them ideally suited for retinal gene therapy, including a lack of pathogenicity, minimal immunogenicity, and the ability to transduce postmitotic cells in a stable and efficient manner. In the sheltered environment of the retina, AAV vectors are able to maintain high levels of transgene expression in the retinal pigmented epithelium (RPE), photoreceptors, or ganglion cells for long periods of time after a single treatment. Each cell type can be specifically targeted by choosing the appropriate combination of AAV serotype, promoter, and intraocular injection site (Dinculescu et al., 2005).

The gene may be provided so as to integrate into the genome of the recipient host cells, or to remain extra-chromosomal. Integration may occur randomly or at precisely defined sites, such as through homologous recombination. In particular, a functional copy of the GPR179 gene may be inserted in replacement of an altered version in a cell, through homologous recombination. Further techniques include gene gun, liposome-mediated transfection, cationic lipid-mediated transfection, etc. Gene therapy may be accomplished by direct gene injection, or by administering ex vivo prepared genetically modified cells expressing a functional GPR179 polypeptide.

Further aspects and advantages of the present invention will be disclosed in the following experimental section, which should be regarded as illustrative and not limiting the scope of the present application.

### EXAMPLES

### Example 1

### Mutations in GPR179 Lead to Autosomal Recessive Complete Congenital Stationary Night Blindness

1.1 Whole Exome Sequencing

To rapidly identify the missing mutations in CSNB cohort 4 exomes from a consanguineous autosomal recessive (ar) cCSNB family (parents who were first cousins, and two of three affected children) and from a sporadic male cCSNB patient of Portuguese origin were sequenced after whole exome enrichment (IntegraGen, Evry, France). One index patient from each family was previously excluded for mutations by Sanger sequencing in GRM6 and TRPM1. In addition, the sporadic male case was also excluded for mutations in NYX. Research procedures were conducted in accordance with institutional guidelines and the Declaration of Helsinki. Prior to genetic testing, informed consent was obtained from all patients and their family members. Ophthalmic examination included best corrected visual acuity, slit lamp examination, fundoscopy, perimetry, full-field electroretinography (ERG) incorporating the ISCEV standards, (Marmor et al, 2009), fundus autofluorescence (FAF) and optical coherence tomography (OCT) (extent of investigation depending on the referring center). Exons of DNA samples were captured using in-solution enrichment methodology (SureSelect Human All Exon Kits Version 3, 7 Agilent, Massy, France) with the company's biotinylated oligonucleotide probe library (Human All Exon v3-50 Mb, Agilent). Each genomic DNA was then sequenced on a sequencer as paired-end 75 bases (Illumina HISEQ, Illumina, San Diego, USA). Image analysis and base calling were performed using Real Time Analysis (RTA) Pipeline version

1.9 with default parameters (Illumina). The bioinformatic analysis of sequencing data was based on a pipeline (CASAVA 1.8, Illumina). CASAVA performs alignment, calls the SNPs based on the allele calls and read depth, and detects variants (SNPs & Indels).

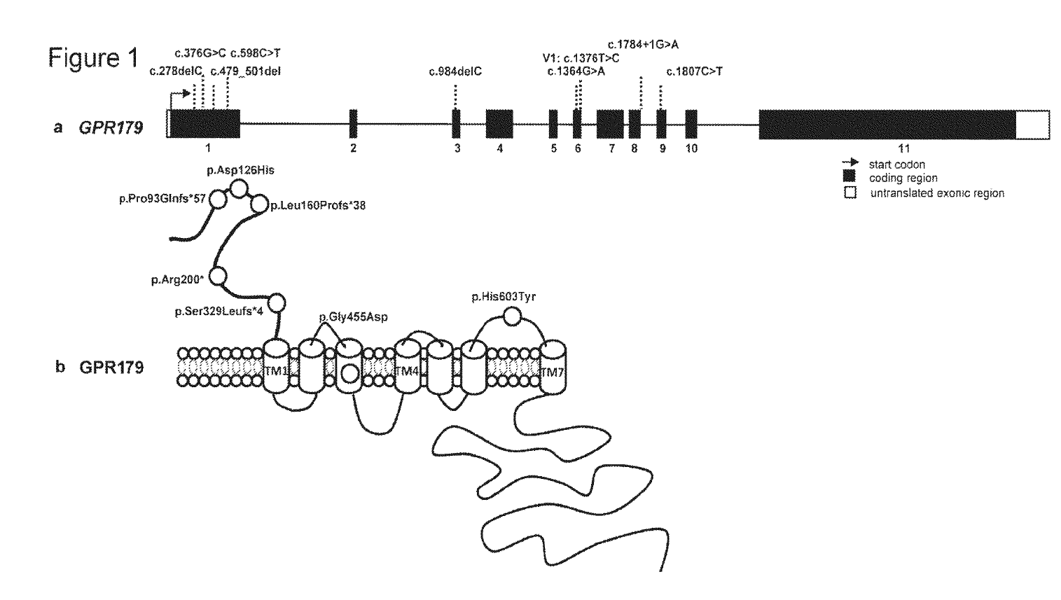

Genetic variation annotation was performed by an in-house pipeline (IntegraGen, Evry, France) and results were provided per sample or family in tabulated text files. After excluding variants observed in dbSNP 132, data were further filtered to only keep variants in coding and splice regions that were present in a homozygous state in the affected children and in a heterozygous state in the parents from the consanguineous family. This allowed the inventors to reduce the number of variants from 5901 Indels to 1 and from 66621 SNPs to 7. The observed deletion represented a repeat deletion in the penultimate exon of VSIG10 and was therefore unlikely to be a disease causing variant. However, three missense mutations predicted to be probably or possibly damaging were identified in three different genes (KIAA0753, CRHR1 (MIM#: 122561) and GPR179 (G-protein coupled receptor 179)) on chromosome 17. The p.Arg518Cys variant found in KIAA0753 was considered unlikely to be disease causing since this arginine residue is not evolutionarily conserved. On the other hand, both the p.Arg259Gln substitution in CRHR1 and the p.His603Tyr in GPR179 affected highly evolutionary conserved amino acid residues (FIG. 1). Interestingly, the other cCSNB case (CICO2756), also studied by whole exome sequencing carried a homozygous 1 by deletion, resulting in a frame shift and premature termination (p.Pro96Glnfs*57) in exon 1 of GPR179. These data strongly support the finding that mutations in GPR179 lead to CSNB found in both families (Table 1).

TABLE 1
Patients with Pathogenic GPR179 Mutations

| | mutations | | | | | | | |
|---|---|---|---|---|---|---|---|---|
| Index Patient, | excluded in | | | | | | Control | |
| Location, Family | following | | | Nucleotide | Allele | | Alleles | |
| Members | genes | Ethnicity | Exon | Exchange | State | Protein Effect | (Mut/WT) | Phenotype Index |
| CIC02756: | NYX, GRM6, | Portuguese- | 1 | c.278delC | hom | p.Pro93Glnfs*57 | 0/366 | cCSNB, |
| male, parents | TRPM1 | French | | | | | | high myopia, nystagmus, |
| far cousins, | | | | | | | | moderate decreased |
| Paris, France | | | | | | | | visual acuity |
| unaff. father | | | 1 | c.278delC | het | p.Pro93Glnfs*57 | | |
| CIC02757 | | | | | | | | |
| unaff. mother | | | 1 | c.278delC | het | p.Pro93Glnfs*57 | | |
| CIC02758 | | | | | | | | |
| CIC03631, | GRM6, | French | 1 | c.376G>C | | p.Asp126His | 0/366 | cCSNB |
| female, Lille, | TRPM1 | | 3 | c.984delC | | Ser329Leufs*4 | 0/372 | high myopia strabismus |
| France | | | | | | | | micro-nystagmus |
| 7699, female, | GRM6, | | 1 | c.479_501del | het | Leu160Profs*38 | 0/366 | cCSNB, strabismus, minimal |
| Tübingen, | TRPM1 | | 6 | c.1364G>A | het | p.Gly455Asp | 0/384 | rotational nystagmus, normal |
| Germany | | | | | | | | visual field |
| unaff. father, | | | 1 | c.479_501del | het | p.Leu160Profs*38 | | |
| 7692 | | | | | | | | |
| unaff. mother, | | | 6 | c.1364G>A | het | p.Gly455Asp | | |
| 7697 | | | | | | | | |
| Y1049; | GRM6, | | 1 | c.598C>T | het | p.Arg200* | 0/378 | cCSNB |
| female, Lille, | TRPM1 | | IVS8 | c.1784+1G>A | het | r.spl? | 0/378 | |
| France | | | | | | | | |
| Y1166, | | | IVS8 | c.1784+1G>A | het | r.spl? | | |
| unaffected father | | | | | | | | |
| Y1167, | | | 1 | c.598C>T | het | p.Arg200* | | |
| unaffected mother | | | | | | | | |
| Y1048, | | | 1 | c.598C>T | het | p.Arg200* | | |
| affected sister | | | IVS8 | c.1784+1G>A | het | r.spl? | | |
| 26985; | GRM6, | Lebanon | 9 | c.1807C>T | hom | p.His603Tyr | 0/366 | cCSNB, left exotropin, |
| Male, | TRPM1 | | | | | | | until age of 2 nystagmus |
| consanguineous | | | | | | | | |
| family, Freiburg, | | | | | | | | |
| Germany, | | | | | | | | |
| father | | | 9 | c.1807C>T | het | p.His603Tyr | | ERG b-wave were slightly |
| CIC03306 | | | | | | | | reduced for high flash strength |
| unaff. mother | | | 9 | c.1807C>T | het | p.His603Tyr | | — |
| CIC03307 | | | | | | | | |
| aff. sister | | | 9 | c.1807C>T | hom | p.His603Tyr | | cCSNB |
| CIC03308 | | | | | | | | |
| aff. sister | | | 9 | c.1807C>T | bom | p.His603Tyr | | cCSNB, visual acuity reduced |
| CIC04005 | | | | | | | | |

Index patients are presented in bold. Abbreviations are as follows: Mut, mutated; het, heterozygous; hom, homozygous; unaff.,. unaffected; aff., affected. CSNB mutations are annotated according to the recommendation of the Human Genome Variation Society, with nucleotide position +1 corresponding to the A of the translation-initiation codon ATG in the cDNA nomenclature RefSeq NM_001004334.2
*26985 Diagnostic: Zurich, Switzerland

For the c.1807C>T p.His603Tyr mutation, both parents were found to be heterozygous, since the nucleotide A was read 11× and 7× in the father and mother respectively, while the G was found 13× and 11×, respectively (reverse strand). The two affected children (CICO2207 and CIC04005) showed 26× and 14× the nucleotide A. The c.278delC deletion detected in the sporadic cCSNB cases was detected 22×; 20 other reads of unknown type were also indicated. This might be due to the fact that at this position multiple C's are present, and thus different reads may occur. Sanger sequencing confirmed the mutations in the index patients of each family. Both mutations co-segregated with the phenotype within the respective family. In addition, NGS data were used to analyze homozygous regions in the affected siblings (CIC03308 and CIC04005) of the consanguineous family. The analysis revealed seven major homozygous regions (>0.5 Mb), which were exclusively present on chromosome 17. GPR179 was present in the second largest homozygous region (10.8 Mb), whereas CRHR1 was present in a smaller region (1.3 Mb). In the other sporadic cCSNB case GPR179 was not present in any major homozygous regions, which can be explained by the fact that the parents were only distant cousins.

The inventors then screened 40 CSNB patients (cCSNB and unclassified CSNB) of various origins and from different clinical centers in Europe, the United States, Canada and Israel using Sanger sequencing for 27 fragments covering the 11 coding exons and flanking intronic regions of GPR179 (RefSeq NM_{—}001004334.2). These were amplified by PCR in the presence of 1.5 mM MgCl2 at an annealing temperature of 60° C. For one of the fragments a specific solution (solution S, 3×, fragment exon 11m, Hot Fire Polymerase, Solis BioDyne, Tartu, Estonia and primers: Table 2) was used.

TABLE 2
Primers to amplify the coding exonic and flanking intronic
regions of GPR179: Ref.:NM_001004334.2

| Name of primer | Primer sequence | SEQ ID NO |
|---|---|---|
| GPR179_Ex1aF | 5′-CTCATACCTACATCCAGAAGC-3′ | SEQ ID NO : 3 |
| GPR179_Ex1aR | 5′-CATCTCCAGAGTAGAGATAAG-3′ | SEQ ID NO : 4 |
| GPR179_Ex1bF | 5′-CAGGATCTGTACCCATGCAG-3′ | SEQ ID NO : 5 |
| GPR179_Ex1bR | 5′-CAGTTCCCAGACAAGTCCTG-3′ | SEQ ID NO : 6 |
| GPR179_Ex1cF | 5′-GAGGATGTGGAATGGTACCAG-3′ | SEQ ID NO : 7 |
| GPR179_Ex1cR | 5′-CAGGCATGCACATGTTCGTG-3′ | SEQ ID NO : 8 |
| GPR179_Ex2F | 5′-GTGCCATAATTCCGAGCATC-3′ | SEQ ID NO : 9 |
| GPR179_Ex2R | 5′-GTATCTCACCTCTGCCAATC-3′ | SEQ ID NO : 10 |
| GPR179_Ex3F | 5′-CAGGTAGGGTTTCCACAGAG-3′ | SEQ ID NO : 11 |
| GPR179_Ex3R | 5′-CTCATACTCAGAAGGTGTAGAC-3′ | SEQ ID NO : 12 |
| GPR179_Ex4F | 5′-GATGTCAGGATGCTCTAGCTG-3′ | SEQ ID NO : 13 |
| GPR179_Ex4R | 5′-CTTGCATCATGCTACACAGTG-3′ | SEQ ID NO : 14 |
| GPR179_Ex5F | 5′-CTGAGTGACCGTTCACATAG-3′ | SEQ ID NO : 15 |
| GPR179_Ex5R | 5′-CAGAGCTCATAGACCAGCTTG-3′ | SEQ ID NO : 16 |
| GPR179_Ex6F | 5′-GTGGTGATGGCTCTATTGCAG-3′ | SEQ ID NO : 17 |
| GPR179_Ex6R | 5′-CACCATTCCATGAGTGAGCTG-3′ | SEQ ID NO : 18 |
| GPR179_Ex7F | 5′-CAGCAATGGACAGAGACCAG-3′ | SEQ ID NO : 19 |
| GPR179_Ex7R | 5′-GAGAACCAGAACAAGAGGAAC-3′ | SEQ ID NO : 20 |
| GPR179_Ex8F | 5′-GTTCCTCTTGTTCTGGTTCTC-3′ | SEQ ID NO : 21 |
| GPR179_Ex8R | 5′-GTGAGGAGTACTGTTAGAGTG-3′ | SEQ ID NO : 22 |
| GPR179_Ex9F | 5′-CTCGTCCTAAGCGTATCAGG-3′ | SEQ ID NO : 23 |
| GPR179_Ex9R | 5′-CAAGTCAGCTCCTAGCTTAG-3′ | SEQ ID NO : 24 |
| GPR179_Ex10F | 5′-GTGAAGCGTCACAAAGTAAGG-3′ | SEQ ID NO : 25 |
| GPR179_Ex10R | 5′-CAGTTTAGGCCAGTGGAGAAG-3′ | SEQ ID NO : 26 |
| GPR179_Ex11aF | 5′-CTGGTCTCACAGGAACAAAGAG-3′ | SEQ ID NO : 27 |
| GPR179_Ex11aR | 5′-GAATTCCGCCAGGTACCTC-3′ | SEQ ID NO : 28 |
| GPR179_Ex11bF | 5′-CACAGGACGAGCTGAAGAAG-3′ | SEQ ID NO : 29 |
| GPR179_Ex11bR | 5′-CTCCTTTGCTTGCCGGTAG-3′ | SEQ ID NO : 30 |
| GPR179_Ex11cF | 5′-CTGCAGAAGTCGCTCAGTG-3′ | SEQ ID NO : 31 |
| GPR179_Ex11cR | 5′-CAGATGTAGGTGAGTAAGTTG-3′ | SEQ ID NO : 32 |
| GPR179_Ex11dF | 5′-CATCAGGCACCAGGTTTCTAC-3′ | SEQ ID NO : 33 |
| GPR179_Ex11dR | 5′-CACACTCTCCTTCTCTCTGTAG-3′ | SEQ ID NO : 34 |
| GPR179_Ex11eF | 5′-GAGAATGAGATGGACGCAGAG-3′ | SEQ ID NO : 35 |
| GPR179_Ex11eR | 5′-GAAACTTGCCTCAGCATGGC-3′ | SEQ ID NO : 36 |
| GPR179_Ex11fF | 5′-CAGGATGCTCCAAGTCTGTC-3′ | SEQ ID NO : 37 |
| GPR179_Ex11fR | 5′-CTCAGATTTGTCTCTGATTCTG-3′ | SEQ ID NO : 38 |
| GPR179_Ex11gF | 5′-CATAGATGTGGTTCCCATGATG-3′ | SEQ ID NO : 39 |
| GPR179_Ex11gR | 5′-CATCACGTTATCATCCAGCTC-3′ | SEQ ID NO : 40 |
| GPR179_Ex11hF | 5′-GTGGCATTGCTGAAGTGTGTC-3′ | SEQ ID NO : 41 |
| GPR179_Ex11hR | 5′-GTCTTGAGGACGTGGTTGTG-3′ | SEQ ID NO : 42 |
| GPR179_Ex11iF | 5′-GTGTCCACAGGAAGATCTCAG-3′ | SEQ ID NO : 43 |
| GPR179_Ex11iR | 5′-GTACCTGGATGCTGAGAACAG-3′ | SEQ ID NO : 44 |
| GPR179_Ex11jF | 5′-CAGGCAAGGTTTCTGCAGATC-3′ | SEQ ID NO : 45 |
| GPR179_Ex11jR | 5′-GTTCTGGGAAGGAACCTGTC-3′ | SEQ ID NO : 46 |
| GPR179_Ex11kF | 5′-CAGCAGCATGAGTAGTGAAGTG-3′ | SEQ ID NO : 47 |
| GPR179_Ex11kR | 5′-CTGCTTCTGTCAGAAGCATCTG-3′ | SEQ ID NO : 48 |
| GPR179_Ex11lF | 5′-GATGTTCCTGATGCAGGTGTG-3′ | SEQ ID NO : 49 |
| GPR179_Ex11lR | 5′-CTGAAGGAACATCTCTCCTTG-3′ | SEQ ID NO : 50 |
| GPR179_Ex11mF | 5′-CAGATGCTTCTGACAGAAGCAG-3′ | SEQ ID NO : 51 |
| GPR179_Ex11mR | 5′-CAGGACAGATGTCTGCCATG-3′ | SEQ ID NO : 52 |
| GPR179_Ex11nF | 5′-GACCCAGAACTCAAAGTCAGC-3′ | SEQ ID NO : 53 |
| GPR179_Ex11nR | 5′-CAATCCCAAGGATAGACAGTG-3′ | SEQ ID NO : 54 |
| GPR179_Ex11oF | 5′-GTGTCAGAGAACTACAAGGAC-3′ | SEQ ID NO : 55 |
| GPR179_Ex11oR | 5′-CTTAGAAGTAGAGTGTCCAGTC-3′ | SEQ ID NO : 56 |

The PCR products were sequenced using a sequencing mix (BigDyeTerm v1.1 CycleSeq kit, Applied Biosystems, Courtaboeuf, France), analyzed on an automated 48-capillary sequencer (ABI 3730 Genetic analyzer, Applied Biosystems) and the results interpreted by applying a software (SeqScape, Applied Biosystems). The inventors detected 3 additional cCSNB patients who carried compound heterozygous disease causing mutations (Table 1). The mutation spectrum identified herein comprises missense, splice site, nonsense mutations and deletions. None of these changes were present in control chromosomes (≧366 chromosomes). For patients whose family members could be investigated, the mutations co-segregated with the cCSNB phenotype and the genotypes were indicative of an autosomal recessive mode of inheritance. Missense mutations were predicted to be pathogenic by PolyPhen and SIFT programs and were also found to affect evolutionarily conserved amino acid residues.

Based on all of the above evidence the inventors conclude that mutations in GPR179 lead to cCSNB.

1.2. Influence of the Mutations

To predict the protein structure and the influence of the mutations identified herein, homology models were created. The human GPR179 sequence (UniProtKB identifier Q6PRD1) was used as a probe for similarity searches in the UniProtKB database with the use of the BlastP program.17,18 In total, more than 100 metazoan sequences (excluding fragments) that were annotated or predicted as GPR179 or GPR158-like were highlighted and aligned with a customized version of the PipeAlign program.19-21 GPR179 codes for a protein with 2367 amino acids that can be divided in 4 main regions corresponding to a small signal peptide (position 1-25), the N-terminal extracellular region (position 26-381), the seven transmembrane (7TM)-spanning region (position 382-628) and the intracellular C-terminal region (position 629-2367) (FIG. 1b). Sequence analysis predicted that the N-terminal extracellular region contains a calcium-binding EGF-like domain (position 278-324) while the C-terminal intracellular is characterized by the presence of a short motif centered on the sequence CPWE which is repeated at least 22 times in the GPR179 related proteins. GPR179 proteins are present in all vertebrates and are closely related to GPR158 and GPR158-like families.

Based on their seven transmembrane domain regions, both families (GPR179 and GPR158) belong to the glutamate receptor or class C family of GPCR. This class includes among others: metabotropic glutamate receptors (GRMs), two γ-aminobutyric acid B receptors (GABABR), the calcium-sensing receptor (CASR), the sweet and umami taste receptors and various orphan receptors (Lagerstrom et al, 2008).

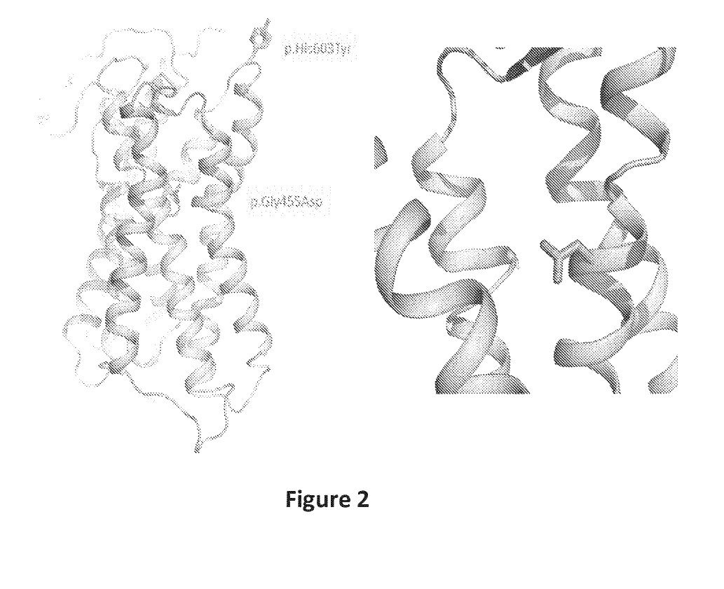

The different deletions and the early termination mutation in GPR179 identified in our patients are located in exon 1 and 3 and are predicted to lead to nonsense-mediated mRNA decay, which may result in the absence of protein product. Alternatively, if a protein is formed, only the first extracellular part would be present lacking all transmembrane domains of GPR179 resulting in truncated protein (FIG. 1b). The missense mutations (p.Asp126Hisp.Gly455Asp and p.His603Tyr) affect evolutionarily conserved amino acid residues, which are predicted to be part of the first extracellular domain, within the third transmembrane domain and in the last extracellular domain (FIG. 1b). Multiple alignment analysis of more than 100 metazoan GPR179-related sequences shows strict conservation of the asparagine at position 126 (Asp126), glycine at position 455 (Gly455) and the histidine at position 603 (His603) in vertebrate sequences. PolyPhen and SIFT programs annotated the three amino acid substitutions to be possibly pathogenic (Ng et al, 2001). These programs use conservation among species and homologs to predict the pathogenic character of a mutation. In addition, an Inductive Logic Programming Prediction web server (Luu et al, 2011) predicted p.Gly455Asp and p.His603Tyr to be pathogenic. This program uses available three-dimensional (3D) structure to predict the influence of a mutation. To date no model of the 3D structure of the amino acid residues <300 is available, therefore the possible pathogenic effect of p.Asp126His could not be predicted using this program. To further gain insight into the deleterious effect of the missense mutations 3D models of the seven transmembrane (7TM)-spanning region of the human GPR179 wild-type and of the 2 mutations (p.Gly455Asp and p.His603Tyr) were generated by homology modeling using MODELLER software (Eswar et al, 2008) (FIG. 2). Two known 7TM templates were used to construct the homology models: the bovine taste receptor (PDB 1F88) and the squid rhodopsine (PDB 2ZIY). For each 3D model construction, 10 homology models were constructed, and the ones with the best normalized DOPE (discrete optimized potential energy) score were selected (Friedrich et al, 2010). The homology 3D models were visualized and analyzed by means of the SM2PH-db28 and figures were constructed with PyMOL software (version 0.99). Multilevel characterization of the mutants (physico-chemical changes and structural modifications induced by the substitution as well as functional and structural features related to the mutated position) can be visualized and analyzed by the MSV3d web server. Structural analysis of the 3D homology models based on the squid RHO 3D model (2ZIY) localized the His603 in the external loop bridging the sixth and seventh trans-membrane while the Gly455 is localized within the third transmembrane helix, which is part of a binding pocket. (FIGS. 1b and 2). The present homology model predicts that the amino acid exchange p.Gly455Asp introduces a long negatively charged side chain which may point towards the cavity of the binding pocket. This suggests that the phenotypic consequences observed for this mutation may be related to some steric constraints hampering the normal functioning of the receptor. The steric constraints in respect to the p.His603Tyr mutation are less obvious. However, strong conservation across species and homologs are indicative for an important role of the histidine at position 603. Although, for the moment the 3D structure of the amino acid residues <300 of GPR179 is not available, it is known from other receptors that the N-terminus of such proteins is important for ligand binding and thus, the p.Asp126His mutation might be associated with loss of this binding. On the other hand, the amino acids that are mutated in the patients might be also important for structural properties of the protein in the endoplasmic reticulum. Thus a misfolded protein is likely to be excluded from the strictly regulated transport to the membrane.

An EST profile is available (Unigene database) and shows a restricted pattern of expression in the human eye, heart and brain. Furthermore, transcriptomic data of whole retina from the rd1 mice, revealed increased expression of GPR179 compared to the wild-type starting from postnatal day 12. The rd1 mouse, carrying Pde6b mutations, is a naturally occurring model with progressive rod photoreceptor degeneration, leading to a complete loss of all rods by post natal day 36, and preserved inner retina.30 This would suggest that GPR179 is expressed in the inner nuclear layer xof the retina. Interestingly, Nyx, another gene, with mutations leading to cCSNB, shows a similar expression profile in the rd1 mouse.

Real-time PCR experiments with two different primer sets (Table 3) confirmed the expression of GPR179 in human retina (commercially available cDNA from Clontech, Saint-Germain-en-Laye, France), giving a signal of ΔCT=13.46 (CTGPR179=30.03) in relation to beta-actin (ACTB MIM: 102630) (CT ACTB=16.57).

TABLE 3
Primers used for the quantitative real-time amplification of
GPR179 transcript: Ref.:NM_001004334.2 and ACTB transcript Ref.:
NM_001101.3

| | | |
|---|---|---|
| GPR179_qPCR_Ex9F | 5′-ACGCTGGCTCTGATCTTCATC-3′ | SEQ ID NO: 57 |
| GPR179_qPCR_Ex10R | 5′-TGTGCTCACTCCAGGCTGAG-3′ | SEQ ID NO: 58 |
| ACTB_qPCR_Ex4F | 5′-CGCCAACACAGTGCTGTCTG-3′ | SEQ ID NO: 59 |
| ACTB_qPCR_Ex5R | 5′-GGAGTACTTGCGCTCAGGAG-3′ | SEQ ID NO: 60 |

Sanger sequencing of the amplified RT-PCR products from retina confirmed the presence of the GPR179 transcript. Using the same conditions, the inventors could not detect the transcript in lymphocytes or HEK293 cells.

The inventors investigated the localization of the Gpr179 protein in adult mouse retina by immunostaining coronal eye cryo-sections with a rabbit polyclonal antibody directed against GPR179 (Sigma-Aldrich, Saint-Quentin Fallavier, France). Bound primary antibody was detected with a secondary antibody (Alexa Fluor 488-conjugated, Invitrogen, Courtaboeuf, France), and the nuclei were counterstained (Hoescht 33258, Sigma-Aldrich).

Immunofluorescence was analyzed using a confocal microscope (FV1000 fluorescent, Olympus, Hamburg, Germany). Gpr179 expression could be detected in the outer plexiform layer (OPL) and in the inner limiting membrane (ILM), in close vicinity with the ganglion cells. Co-localization studies with a mouse anti-Bassoon antibody (Enzo Lifescienes, Lyon, France), a specific marker for ribbon synapse, excluded a close vicinity of Gpr179 with pre-synaptic terminals. Furthermore, immunostaining with mouse antibodies against Goa (Millipore, Molsheim, France) and PKCα (Sigma-Aldrich), two specific ON-bipolar markers, demonstrated the absence of co-localization of Gpr179 with these proteins. Instead, Gpr179 appears to be localized in a distinct compartment within bipolar cells or in other cells such as horizontal cells. Interestingly, bipolar cell dendrites, stained with PKCa seem to surround Gpr179. Alternatively, the Gpr179 OPL staining could also be localized within Midler cell processes present within this layer. In addition, a mouse antibody against Calretinin (Millipore), a specific marker for ganglion cells and their dendrites, was used and did not show colocalization with, Gpr179 immunostaining. Instead, Gpr179 was highly expressed in Midler cell endfeet at the level of the ILM, as it had previously been shown for the potassium channel Kir 4.1 macromolecular complex.31-33

Therefore, immunolocalization of Gpr179 suggests its localization in the OPL either in bipolar cells in a cellular compartment distinct from synaptic membrane and cell body and/or in horizontal cells and/or in Müller cells processes as well as within the Müller cell endfeet. The OPL localization of Gpr179 and the same associated ON-bipolar dysfunction phenotype as for Grm6, Nyx or Trpm1 mutations34-38 would suggest that GPR179 is part of the same transduction pathway and could directly interact with any of these proteins. However, immunolocalization studies are not in keeping with this hypothesis. Instead, immunostaining suggesting Müller cells localization could place Gpr179 functional role within these cells, possibly through the Kir4.1 macromolecular complex. This complex was shown to involve at least the potassium channel Kir4.1, the water channel aquaporin-4 (AQP4) and the dystrophin isoform Dp71. Interestingly, although Kir4.1 and Agp4 knock-out mice do not show Schubert-Bornshein ERG abnormalities,39,40 a subset of dystrophin mutations, responsible for Duchenne muscular dystrophy (DMD MIM#: 310200).) are associated with such ERG abnormalities.41-44 Therefore, one hypothesis would be that Gpr179 is part of the Kir4.1 macromolecular complex. Gpr179 may directly interact with dystrophin isoforms, and its dysfunction would lead to cCSNB in a similar mechanism as in DMD. In order to reconcile the Grm6/Nyx/Trpm1 signaling pathway within bipolar cells and Gpr179 within Muller cells, one may hypothesize that Gpr179 may yet be involved in an unknown interaction between ON-bipolar cells and Müller cells which would be essential for ON-bipolar cell depolarization resulting in b-wave formation. On the other hand, our immunostaining studies could also suggest specific localization of Gpr179 within horizontal cells. Therefore, another hypothesis could be that ON-bipolar cells directly interact with horizontal cells. Lack of this interaction due Gpr179 dysfunction could lead to the reduced b-wave observed in patients with cCSNB.

### Example 2

The GPR179 mutation spectrum leading to cCSNB compromises deletions, nonsense mutations, splice site and missense mutations. Even though the underlying pathogenic mechanism for the truncating mutations is estimated to be complete loss of functional GPR179, the impact of the missense mutations could be either due to mislocalization of the protein, absence of ligand binding or loss of interaction with other proteins important for signaling from photoreceptor to bipolar cells.

It has been shown that mouse Gpr179 transcript is expressed in the upper part of the inner cells, presumably in bipolar cells and that the human orthologue localizes in the tips of the dendrites of bipolar cells in human retina.

In the present study, it was investigated the impact of the missense mutations by performing life cells staining and subsequent intracellular staining of normal and mutated GPR179 protein in vitro.

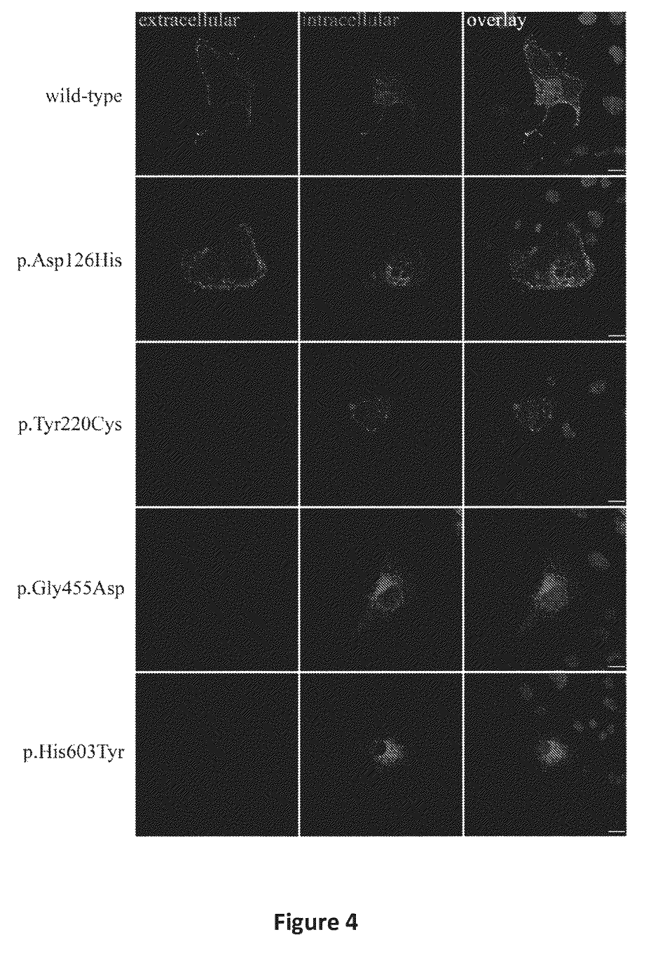

While the normal GPR179 could be detected at the cell surface of COS-1 cells overexpressing the protein, three of four missense mutations p.Tyr220Cys, p.Gly455Asp and p.His603Tyr could be only detected in intracellular compartments (FIG. 4).

Expression Constructs

The coding DNA sequence and BamHI and NotI-linkers of the normal and mutated human GPR179 gene were synthesized in an optimized way and cloned in an expression vector (pcDNA3, Invitrogen, Courtaboeuf, France) by a company (GeneCust). To validate a commercially available human anti-GPR179 antibody (HPA017885-100UL, Sigma-Aldrich), we inserted in frame a flag-tag between the predicted signal sequence (after amino acid 26) and the main sequence. The sequence of the respective plasmids were verified by the company and in our laboratory by Sanger sequencing using standard conditions on an automated 48-capillary sequencer (BigDyeTerm v1.1 CycleSeq kit, ABI 3730 Genetic analyzer, Applied Biosystems, Courtaboeuf, France) with specific primers designed against the normal and optimized synthetic GPR179 sequence (Table 4) and vector oligonucleotides (using standard T7, SP6 and BGH oligonucleotides).

TABLE 4

| Name of primer | Primer sequence |
|---|---|
| pGRP179seq1R | 5′-GAGTGTCGCCTACATAACCG-3′ |
| | SEQ ID NO: 61 |
| pGRP179seq1F | 5′-CGGTTATGTAGGCGACACTC-3′ |
| | SEQ ID NO: 62 |
| pGRP179seq2F | 5′-GCTGTACTAGCTGCATGGAC-3′ |
| | SEQ ID NO: 63 |
| pGRP179seq3F | 5′-GCTTCCTGGCTGTATGGAC-3′ |
| | SEQ ID NO: 64 |
| pGRP179seq4F | 5′-GAGGATGAGCTGGATCTGC-3′ |
| | SEQ ID NO: 65 |
| pGRP179seq5F | 5′-GTCGTCGTCTGCTGAGCAG-3′ |
| | SEQ ID NO: 66 |
| pGRP179seq6F | 5′-GTGCTCCGCTGTCTGCTC-3′ |
| | SEQ ID NO: 67 |
| pGRP179seq7F | 5′-CCGTGCTGGCGAAAACGAG-3′ |
| | SEQ ID NO: 68 |
| pGRP179seq8F | 5′-GAACGCAAAGCAGAACGCG-3′ |
| | SEQ ID NO: 69 |
| pGRP179seq9F | 5′-GTATGTCCTTGGGAATCCGC-3′ |
| | SEQ ID NO: 70 |
| pGRP179seq10F | 5′-GTTTGCCTGTGGGAGGCG-3′ |
| | SEQ ID NO: 71 |
| pGRP179seq11F | 5′-GAATGGACCTCTGCGCAGG-3′ |
| | SEQ ID NO: 72 |
| pGRP179seq12F | 5′-GCGGTTACTGCACCGGAG-3′ |
| | SEQ ID NO: 73 |
| pGRP179seq13F | 5′-GCACCGAAGATCAGCGACC-3′ |
| | SEQ ID NO: 74 |
| GPR179_Ex11eF | 5′-GAGAATGAGATGGACGCAGAG-3′ |
| | SEQ ID NO: 35 |
| GPR179_Ex11iF | 5′-GTGTGTCCACAGGAAGATCTCAG-3′ |
| | SEQ ID NO: 79 |
| GPR179_Ex11nF | 5′-GACCCAGAACTCAAAGTCAGC-3′ |
| | SEQ ID NO: 53 |

Cell Culture, Transfection and Immunofluorescence

Transient transfection studies were performed in COS-1 cells. In 24-well plates, at the presence of coated cover slips, 130000 cells per well were seeded and transfected after 6 h with 10 μg human normal and mutated GPR179 plasmid with the Calcium phosphate method (Bacchetti et al., 1977).

To validate the human anti-GPR179 antibody mentioned above in vitro, cells were permeabilized after 36 h transfection and stained for intracellular GPR179 protein, by using the human anti-GPR179 antibody and mouse anti-Flag antibody (M2 F3165, Sigma-Aldrich) in the same experiment, which were visualized with donkey anti-rabbit Cy3 (Jackson Immuno research Laboratories) and anti-mouse Alexa 488 (Jackson Immuno research Laboratories) antibodies. To investigate the localization of normal and mutated GPR179, life cell extracellular staining and subsequent intracellular staining was performed as. Just briefly extracellular GPR179 protein was detected by life cell staining using rabbit anti-GPR179 and anti-rabbit Alexa 488 (Jackson Immuno research Laboratories) antibodies. Subsequently, after fixation of stained extracellular protein, intracellular protein was detected by rabbit anti-GPR179 and anti-rabbit Cy3 (Jackson Immuno research Laboratories) antibodies. Stained cells were analyzed with a fluorescence or confocal microscope (FV1000 fluorescent, Olympus, Hamburg, Germany).

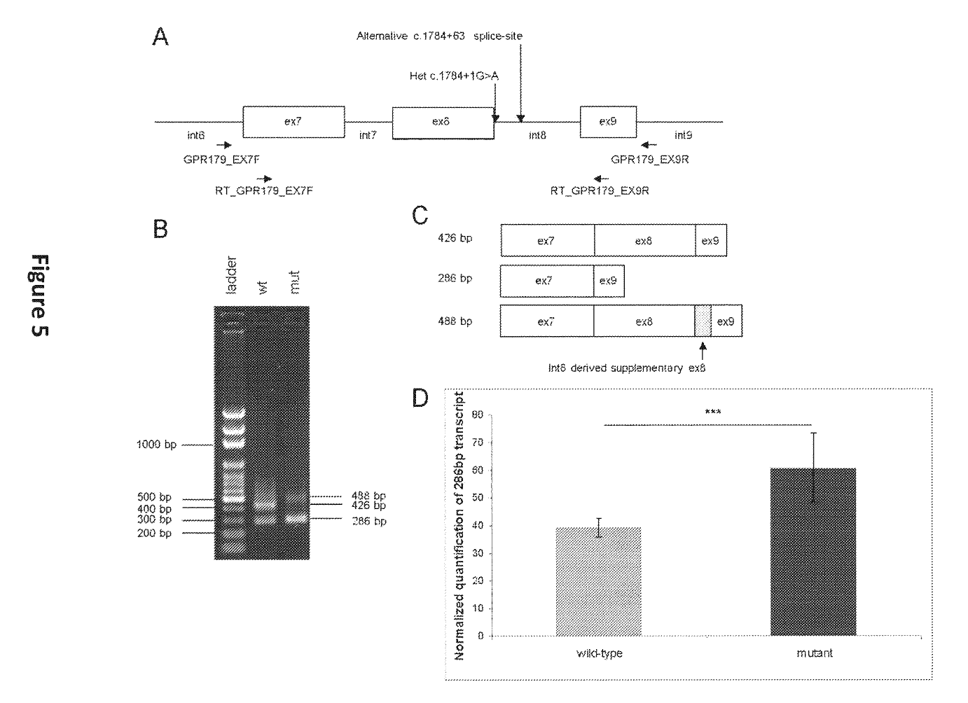

The mini-gene-approach in COS-1 cells overexpressing exons 7, 8 and 9 revealed two different spliced products compared to control (FIG. 5).

Mini-Gene-Approach

Patient genomic DNA containing the heterozygous c.1784+1G>A mutation has been amplified between intron 6 and intron 9 with GPR179 oligonucleotides used for the initial mutation screening (GPR179_EX7F and GPR179_EX9R) with a DNA Polymerase (HOT FIREPol, Solys Biodine, Tartu, Estonia). The amplicon was subcloned in a vector (pCRII-TOPO vector, Invitrogen) and normal and plasmids containing the expected splice site mutation have been identified by Sanger sequencing using standard M13 oligonucleotides and were cloned into a vector (pBudCE4.1 vector, Invitrogen) using the HindIII and XbaI restriction sites. Transient transfection studies were performed in COS-1 cells in 6 wells plates and total RNA has been extracted using the RNeasy Mini Kit (Qiagen, Hilden, Germany). Reverse transcription have been performed using a Reverse Transcriptase (SuperscriptII, Invitrogen). To validate the in vitro splicing products, a PCR has been performed with oligonucleotides in exon 7 and 9 of GPR179 (RT_GPR179_EX7F 5′GTGCTGCAGCTGTTTCTGTC3′ SEQ ID NO:75, and RT_GPR179_EX9R 5′ AAGAGGAGGAGGGTCCAGTC3′ SEQ ID NO:76). Five μL of the RT-PCR products were investigated by electrophorese on a 2% agarose gel, 1 μL was cloned in a vector (pCRII-TOPO, Invitrogen) and Sanger sequenced using standard M13 oligonucleotides. To normalize GPR179 RT-PCR values, a beta-actine PCR (using ACTNBqPCR Ex4F CGCCAACACAGTGCTGTCTG SEQ ID NO:77, and ACTNB_qPCR_Ex5R GGAGTACTTGCGCTCAGGAG SEQ ID NO:78 primers) were performed on the obtained cDNA and were investigated like previously described above the GPR179 minigene RT-PCR. All PCR experiments were performed and carried out five times. Negative controls were included. An assessment of GPR179 mRNA and beta-actine mRNA levels were performed by a semi-quantitative analysis using ChemiDoc XRS and Quantity One version 4.4.0 software (Bio-Rad, Hercules, Calif., USA).

Statistical Analyses

Statistical analyses were performed by using the SPSS® statistical software version 19.0 (SPSS, Inc, Chicago, Ill.). An assessment of normality for GPR179 mRNA levels was performed prior to applying the required statistical test.

For the study of c.1784+1G>A mutation influence on GPR179 mRNA levels, mean comparisons between groups (WT and mutated) were analysed by paired samples t-tests. The threshold for statistical significance was set at P<0.05.

Together, these findings indicate that GPR179 acts in the same pathway such as GRM6, NYX and TRPM1, other molecules implicated in the same phenotype of CSNB and that not only protein truncating mutations but at least three GPR179 missenses and one splice site mutation are implicated in the complete loss of GPR179 protein function, explaining the severely reduced scotopic b-wave.

The pathogenic mechanism of missense mutation p.Asp126His needs still to be elucidated. Although, for the moment, the 3D structure of the amino acid residues <300 of GPR179 is not available, we know from other receptors that the N-terminus of such G-protein coupled receptors is important for ligand binding, and thus the p.Asp126His mutation might be associated with loss of this binding, a hypothesis which may open a way to treat patients with such a mutation.

### REFERENCES

Audo, I., Robson, A. G., Holder, G. E., and Moore, A. T. (2008). The negative ERG: clinical phenotypes and disease mechanisms of inner retinal dysfunction. Surv. Ophthalmol. 53, 16-40.
Audo, I., Kohl, S., Leroy, B. P., Munier, F. L., Guillonneau, X., Mohand-Said, S., Bujakowska, K., Nandrot, E. F., Lorenz, B., Preising, M., et al. (2009). TRPM1 is mutated in patients with autosomal-recessive complete congenital stationary night blindness. Am. J. Hum. Genet. 85, 720-729.
Bacchetti, S. and Graham, F. L. (1977) Transfer of the gene for thymidine kinase to thymidine kinase-deficient human cells by purified herpes simplex viral DNA. Proc. Natl. Acad. Sci. U.S.A., 74, 1590-4
Bech-Hansen, N. T., Naylor, M. J., Maybaum, T. A., Sparkes, R. L., Koop, B., Birch, D. G., Bergen, A. A., Prinsen, C. F., Polomeno, R. C., Gal, A., et al. (2000). Mutations in NYX, encoding the leucine-rich proteoglycan nyctalopin, cause X-linked complete congenital stationary night blindness. Nat. Genet. 26, 319-323.
Dinculescu et al., Hum Gene Ther. 2005 June;16(6):649-63
Dryja, T. P., McGee, T. L., Berson, E. L., Fishman, G. A., Sandberg, M. A., Alexander, K. R., Derlacki, D. J., and Rajagopalan, A. S. (2005). Night blindness and abnormal cone electroretinogram ON responses in patients with mutations in the GRM6 gene encoding mGluR6. Proc. Natl. Acad. Sci. U.S.A. 102, 4884-4889.
Dvir, L., Srour, G., Abu-Ras, R., Miller, B., Shalev, S. A., and Ben-Yosef, T. (2010). Autosomal-recessive early-onset retinitis pigmentosa caused by a mutation in PDE6G, the gene encoding the gamma subunit of rod cGMP phosphodiesterase. Am. J. Hum. Genet. 87, 258-264.
Eswar, N., Eramian, D., Webb, B., Shen, M. Y., and Sali, A. (2008). Protein structure modeling with MODELLER. Methods Mol. Biol. 426, 145-159.
Friedrich, A., Gamier, N., Gagniere, N., Nguyen, H., Albou, L. P., Biancalana, V., Bettler, E., Deleage, G., Lecompte, O., Müller, J., et al. (2010). SM2PH-db: an interactive system for the integrated analysis of phenotypic consequences of missense mutations in proteins involved in human genetic diseases. Hum. Mutat. 31, 127-135.
Lagerstrom, M. C., and Schioth, H. B. (2008). Structural diversity of G protein-coupled receptors and significance for drug discovery. Nat. Rev. Drug Discov. 7, 339-357.
Li, Z., Sergouniotis, P. I., Michaelides, M., Mackay, D. S., Wright, G. A., Devery, S., Moore, A. T., Holder, G. E., Robson, A. G., and Webster, A. R. (2009). Recessive mutations of the gene TRPM1 abrogate ON bipolar cell function and cause complete congenital stationary night blindness in humans. Am. J. Hum. Genet. 85, 711-719.
Luu, T., Nguyen, N., Friedrich, A., Muller, J., Moulinier, L., and Poch, O. (2011). Extracting knowledge from a mutation database related to human monogenic disease using inductive logic programming. International Conference on Bioscience, Biochemistry and Bioinformatics; Singapore. IACSIT 5, 83-100.
Marmor, M. F., Fulton, A. B., Holder, G. E., Miyake, Y., Brigell, M., and Bach, M. (2009). ISCEV Standard for full-field clinical electroretinography (2008 update). Doc. Ophthalmol. 118, 69-77.
Miyake, Y., Yagasaki, K., Horiguchi, M., Kawase, Y., and Kanda, T. (1986). Congenital stationary night blindness with negative electroretinogram. A new classification. Arch. Ophthalmol. 104, 1013-1020.
Morgans, C. W., Ren, G., and Akileswaran, L. (2006). Localization of nyctalopin in the mammalian retina. Eur. J. Neurosci. 23, 1163-1171.
Nakamura, M., Sanuki, R., Yasuma, T. R., Onishi, A., Nishiguchi, K. M., Koike, C.,
Kadowaki, M., Kondo, M., Miyake, Y., and Furukawa, T. (2010). TRPM1 mutations are associated with the complete form of congenital stationary night blindness. Mol. Vis. 16, 425-437.
Ng, P. C., and Henikoff, S. (2001). Predicting deleterious amino acid substitutions. Genome Res. 11, 863-874.
Nishiguchi, K. M., Sandberg, M. A., Kooijman, A. C., Martemyanov, K. A., Pott, J. W., Hagstrom, S. A., Arshaysky, V. Y., Berson, E. L., and Dryja, T. P. (2004). Defects in RGS9 or its anchor protein R9AP in patients with slow photoreceptor deactivation. Nature 427, 75-78.
Peachey N S, Ray T A, Florijn R, Rowe L B, Sjoerdsma T, Contreras-Alcantara S, Baba K, Tosini G, Pozdeyev N, Iuvone P M, et al. (2012). GPR179 is required for depolarizing bipolar cell function and is mutated in autosomal-recessive complete congenital stationary night blindness, Am J Hum Genet, 90, 331-339
Pusch, C. M., Zeitz, C., Brandau, O., Pesch, K., Achatz, H., Feil, S., Scharfe, C., Maurer, J., Jacobi, F. K., Pinckers, A., et al. (2000). The complete form of X-linked congenital stationary night blindness is caused by mutations in a gene encoding a leucine-rich repeat protein. Nat. Genet. 26, 324-327.
Van Genderen, M. M., Bijveld, M. M., Claassen, Y. B., Florijn, R. J., Pearring, J. N., Meire, F. M., McCall, M. A., Riemslag, F. C., Gregg, R. G., Bergen, A. A., et al. (2009). Mutations in TRPM1 are a common cause of complete congenital stationary night blindness. Am. J. Hum. Genet. 85, 730-736.
Wycisk, K. A., Zeitz, C., Feil, S., Wittmer, M., Forster, U., Neidhardt, J., Wissinger, B., Zrenner, E., Wilke, R., Kohl, S., et al. (2006). Mutation in the auxiliary calcium-channel subunit CACNA2D4 causes autosomal recessive cone dystrophy. Am. J. Hum. Genet. 79, 973-977.
Zeitz, C., van Genderen, M., Neidhardt, J., Luhmann, U. F., Hoeben, F., Forster, U., Wycisk, K., Matyas, G., Hoyng, C. B., Riemslag, F., et al. (2005). Mutations in GRM6 cause autosomal recessive congenital stationary night blindness with a distinctive scotopic 15-Hz flicker electroretinogram. Invest. Ophthalmol. Vis. Sci. 46, 4328-4335.
Zeitz, C., Kloeckener-Gruissem, B., Forster, U., Kohl, S., Magyar, I., Wissinger, B., Matyas, G., Borruat, F. X., Schorderet, D. F., Zrenner, E., et al. (2006). Mutations in CABP4, the gene encoding the Ca2+-binding protein 4, cause autosomal recessive night blindness. Am. J. Hum. Genet. 79, 657-667.
Zeitz, C., Labs, S., Lorenz, B., Forster, U., Uksti, J., Kroes, H. Y., De Baere, E., Leroy, B. P., Cremers, F. P., Wittmer, M., et al. (2009). Genotyping microarray for CSNB-associated genes. Invest. Ophthalmol. Vis. Sci. 50, 5919-5926.

1. An in vitro method for diagnosing an autosomal recessive complete congenital stationary night blindness (cCSNB) in a subject, which method comprises determining the presence of an alteration in the GPR179 gene in a biological sample of said subject.

2. An in vitro method for determining the risk for a subject to transmit an autosomal recessive complete congenital stationary night blindness (cCSNB) to his/her progeny, which method comprises determining the presence of an alteration in the GPR179 gene in a biological sample of said subject.

3. The method of claim 1, wherein said subject has been diagnosed with a congenital stationary night blindness (CSNB) or is related to an individual, e.g. a sibling or a parent, with a CSNB.

4. The method of claim 1, wherein the alteration is a mutation, deletion, or addition of one or more nucleotides in at least one exon of the GPR179 gene, or in splicing donor or acceptor site.

5. The method of claim 4, wherein the alteration is selected from the group consisting of
i. a deletion of nucleotide C at position 278 on SEQ ID NO:1;
ii. a substitution of nucleotide G into C at position 376 on SEQ ID NO:1;
iii. a deletion of nucleotides 479 to 501 on SEQ ID NO:1;
iv. a substitution of nucleotide C into T at position 598 on SEQ ID NO:1;
v. a deletion of nucleotide C at position 984 on SEQ ID NO:1;
vi. a substitution of nucleotide G into A at position 1364 on SEQ ID NO:1;
vii. a substitution of nucleotide G into A at position 1784+1 on SEQ ID NO:1; and
viii. a substitution of nucleotide C into T at position 1807 on SEQ ID NO: 1.

6. The method of claim 1, wherein the presence of an alteration in the GPR179 gene is determined by sequencing, selective hybridization and/or selective amplification.

7. The method of claim 1, wherein the presence of an alteration in the GPR179 gene is determined by detecting a mutation in the amino acid sequence of the protein encoded by said gene.

8. The method of claim 1, wherein the presence of an alteration in the GPR179 gene is determined by determining the level of expression of the GPR179 protein in a biological sample of the subject, wherein an absence of expression or a decreased level of expression the GPR179 protein with respect to a healthy control is indicative of a cCSNB.

9. The method of claim 1, which further comprises determining the presence of an alteration in at least one of the following genes: NYX, CACNA1F, GRM6, TRPM1, CABP4, CACNA2D4, SLC24A1, RHO, GNAT1 and PDE6B.

10. An in vitro method of selecting compounds as candidate medicaments for treating autosomal recessive cCSNB, said method comprising contacting a test compound with a GPR179 protein or gene or a fragment thereof and determining the ability of said test compound to bind the GPR179 protein or gene or a fragment thereof.

11. An in vitro method of selecting compounds as candidate medicaments for treating autosomal recessive cCSNB, said method comprising contacting a test compound with a recombinant host cell expressing a GPR179 protein, and determining the ability of said test compound to bind said GPR179 protein and to modulate the activity of GPR179 protein.

12. An in vitro method of selecting compounds as candidate medicaments for treating autosomal recessive cCSNB, said method comprising contacting a test compound with a GPR179 gene and determining the ability of said test compound to modulate the expression of said gene.

13. An in vitro method of selecting compounds as candidate medicaments for treating autosomal recessive cCSNB, said method comprising contacting a test compound with a recombinant host cell comprising a reporter construct, said reporter construct comprising a reporter gene under the control of a GPR179 gene promoter, and selecting the test compounds that modulate expression of the reporter gene.

14. The method according to claim 10, wherein said GPR179 protein or gene or a fragment thereof is an altered or mutated a GPR179 protein or gene or a fragment thereof comprising the alteration or mutation.

15. The use of a compound selected from the group consisting of a nucleic acid encoding GPR179 protein or a ligand, preferably an agonist, of GPR179, in the manufacture of a pharmaceutical composition for treating autosomal recessive cCSNB in a subject.

16. The method of claim 2, wherein said subject has been diagnosed with a congenital stationary night blindness (CSNB) or is related to an individual, e.g. a sibling or a parent, with a CSNB.

17. The method of claim 2, wherein the alteration is a mutation, deletion, or addition of one or more nucleotides in at least one exon of the GPR179 gene, or in splicing donor or acceptor site.

18. The method of claim 17, wherein the alteration is selected from the group consisting of
i. a deletion of nucleotide C at position 278 on SEQ ID NO:1;
ii. a substitution of nucleotide G into C at position 376 on SEQ ID NO:1;
iii. a deletion of nucleotides 479 to 501 on SEQ ID NO:1;
iv. a substitution of nucleotide C into T at position 598 on SEQ ID NO:1;
v. a deletion of nucleotide C at position 984 on SEQ ID NO:1;
vi. a substitution of nucleotide G into A at position 1364 on SEQ ID NO:1;
vii. a substitution of nucleotide G into A at position 1784+1 on SEQ ID NO:1; and
viii. a substitution of nucleotide C into T at position 1807 on SEQ ID NO: 1.

19. The method of claim 2, wherein the presence of an alteration in the GPR179 gene is determined by sequencing, selective hybridization and/or selective amplification.

20. The method of claim 2, which further comprises determining the presence of an alteration in at least one of the following genes: NYX, CACNA1F, GRM6, TRPM1, CABP4, CACNA2D4, SLC24A1, RHO, GNAT1 and PDE6B.

21. The method according to claim 11, wherein said GPR179 protein or gene or a fragment thereof is an altered or mutated a GPR179 protein or gene or a fragment thereof comprising the alteration or mutation.

22. The method according to claim 12, wherein said GPR179 protein or gene or a fragment thereof is an altered or mutated a GPR179 protein or gene or a fragment thereof comprising the alteration or mutation.

23. The method according to claim 13, wherein said GPR179 protein or gene or a fragment thereof is an altered or mutated a GPR179 protein or gene or a fragment thereof comprising the alteration or mutation.
